SEQUENCE LISTING (1 sequence)

SEQ ID NO: 1 - protein, 315 aa, Homo sapiens
ASRLLLNNGAKMPILGLGTWKSPPGQVTEAVKVAIDVGYRHIDCAHVYQNENEVGVAIQEKLREQVVKREELFIVSKLWCTYHEKGLVKGACQKTLSDLKLDYLDLYLIHWPTGFKPGKEFFPLDESGNVVPSDTNILDTWAAMEELVDEGLVKAIGISNFNHLQVEMILNKPGLKYKPAVNQIECHPYLTQEKLIQYCQSKGIVVTAYSPLGSPDRPWAKPEDPSLLEDPRIKAIAAKHNKTTAQVLIRFPMQRNLVVIPKSVTPERIAENFKVFDFELSSQDMTTLLSYNRNWRVCALLSCTSHKDYPFHEEF

(19) United States
(12) Patent Application Publication
(10) Pub. No.: US 2005/0004225 A1
(43) Pub. Date: Jan. 6, 2005

(54) Oxidoreductase inhibitors and methods of screening and using thereof

(76) Inventor: Ganesaratnam K. Balendiran, Arcadia, CA (US)

Correspondence Address: PERKINS COIE LLP, POST OFFICE BOX 1208, SEATTLE, WA, 98111-1208, US

(21) Appl. No.: 10/826,679

(22) Filed: Apr. 16, 2004

Related U.S. Application Data
(60) Provisional application No. 60/463,629, filed on Apr. 16, 2003.

(51) Int. Cl. A61K 31/19

(52) U.S. Cl. 514/571

(57) ABSTRACT

The present invention relates to 1) the design and synthesis of analogs to glutathione conjugates which bind to or interact with aldose reductase (AR) through unique conformations that are distinctly different from the substrates and inhibitors of AR which are members of sugar metabolism; 2) the screening of the analogs to identify those that interact with or inhibit or enhance the activity of AR; and 3) the use of AR ligands, AR inhibitors (AR antagonsits) or AR enhancer (AR agonists) in the detection of AR activity, the modulation of AR activity, and the treatment of conditions in a subject in need of modulating AR activity. Such conditions include but not limited to cardiovascular disease, diabetes, artheriosclerosis, cancer, neoplasm, obesity, cataract, retinopathy, keratopathy, nephropathy, neurosis, thrombosis, faulty union of corneal injury and neuropathy. Examples of the treatment include the use of fibrates as AR inhibitors to treat these conditions.

### CROSS-REFERENCE TO RELATED APPLICATION

This application claims priority to U.S. Provisional Patent Application No. 60/463,629 filed Apr. 16, 2003, which is incorporated herein in its entirety by reference, including drawings.

### FIELD OF THE INVENTION

The present invention relates to aldose reductase as a member of oxidoreductase. In particular, the present invention is directed to designing and screening compounds that are analogs to glutathione conjugates or otherwise which bind to a specific binding site of aldose reductase, identifying a new set of aldose reductase inhibitor or enhancers (modulators), and using the aldose inhibitors or enhancers in the treatment of diseases such as Atherosclerosis, cancer, cardiovascular diseases, obesity, stroke and diabetes.

### BACKGROUND OF THE INVENTION

The oxidoreductase comprise a family of Mᵣ˜36,000 proteins that catalyze the reduction of a wide variety of substrates including aliphatic and polycyclic aldehydes, aldoses, lipid-derived aldehydes, and xenobiotics. Aldose reductase (AR) is a member of the oxidoreductase family. AR catalyzes the NADPH-dependent reduction of glucose to sorbitol, the first step of the sorbitol pathway. The pathway is completed by sorbitol dehydrogenase, which catalyzes the NAD⁺-dependent oxidation of sorbitol to fructose. A large body of evidence, derived principally from experimental animal studies, supports the hypothesis that enhanced metabolism of glucose through the AR-catalyzed polyol pathway results in biochemical imbalances associated with diabetic complications (1,2, 56, 95). Due to its involvement in the pathogenesis of diabetic complications, AR and its inhibitors are well studied in the polyol pathway. Complex crystal structures of AR are available for sorbitol, fidarestat, zopolrestat, tolrestat, sorbinil, citrate, cacodylate, glucose 6-phosphate, oxazolecarbamate, WF-3681 and IDD384 in more than one crystal form in the protein data bank (3-12).

The demonstration that AR catalyzes the reduction of lipid aldehydes and their conjugates with glutathione, and that the activity of AR is enhanced by growth factors, as observed in vascular smooth muscle cells, raises the possibility that AR may be involved in cellular functions in addition to glucose metabolism (15). Studies have shown that AR is broad specificity aldehyde reductase and that unsaturated aldehydes, such as those derived from lipid peroxidation are excellent substrates of this enzyme (13-19) and indicated that AR is part of the cellular defenses against aldehyde toxicity. For example, AR has been shown to detoxify daunorubicin (29). Studies also have shown that AR is upregulated in giant cell arteritis, an inflammatory vasculopathy sickness that affects medium-sized arteries, indicating that AR has a role in inflammation (30).

It has been reported that AR efficiently catalyzes the reduction of medium and long chain saturated aldehydes, with Kₘ values considerably lower than that for short chain aldehydes (18). AR is reported to be involved in the reduction of unsaturated aldehydes such as 4-hydroxy-trans-2-nonenal (HNE) as well as their glutathione conjugates (15, 42, 44). Interestingly, in the case of short chain aldehydes, such as acrolein, conjugation with glutathione led to a 100-fold increase in the catalytic efficiency of the enzyme. This increase in efficiency by glutathiolation is evident for aldehydes of diverse chemical structure, although the extent of catalytic enhancement was dependent upon the chain length of the aldehyde. It has been demonstrated by structure function studies an important role of AR in the metabolism of glutathione conjugates of endogenous and xenobiotic aldehydes (16). In addition, studies have shown that alterations of the functionality and structure of glutathione resulted in diminished catalytic efficiency in the reduction of the acrolein adduct indicating the substrate specificity of AR (16).

Recent studies also shows that AR plays a role in carcinogenesis. Tumor-associated protein variant (35 kDa/pI 7.4) was identified in rat hepatocarcinogenesis (26). This protein is expressed in the liver during embryogenesis, but absent in adult rat liver. However, it is re-expressed and functionally active during liver carcinogenesis (27). This protein has been shown to share 98.5% amino acid sequence identity with rat lens AR indicating hepatoma-derived AR like protein and rat lens AR are related proteins encoded by different genes (26). It has been shown that the hepatoma-derived, AR-like protein is already expressed in the preneoplastic stage of hepatocarcinogenesis and might potentially serve as a marker enzyme in early neoplasia. About 29% of human liver cancers overexpress AR and about 54% of them overexpress an AR-like protein whose amino acid sequence is 70% identical to that of AR (28). It is well known that liver cancer hepatocellular carcinoma (HCC) is resistant to a number of anticancer drugs reducing its efficacy. Interestingly, overexpression of AR makes the cells more resistant to cancer chemotherapeutic drugs (68). Expression of novel AR-related protein in all five tested cancer cell lines suggests that AR may play an important role in liver carcinogenesis (69, 70).

While glutathione conjugates are efficient substrates for AR (42), the conjugates and glutathione play a key role in the determining the sensitivity of cancer cells to radiation and drug-induced cytoxicity (81, 82, 74). Glutathione level and its redox status are studied in (a) the presence of alkylating agent melphalan (L-PAM) in a series of DU-145 prostate carcinoma cell lines (107), (b) cisplatin-resistant ovarian carcinoma cell lines (108), (c) bronchial carcinoma cell line A-427 (109), (d) malignant breast tissues and blood, (e) colon cancers and (f) Ehrlich ascites tumor-bearing mice. Elevation of intracellular glutathione levels is associated with mitogenic stimulation (83), regulation of DNA synthesis (84), control of tumor-cell proliferation by regulating protein kinase C activity (95, 119) and intracellular pH (85). The onset of severe tumor-related weight loss (cachexia) in the host is accompanied by a decrease in the rate of cancer cell proliferation and a decrease in glutathione in the tumor (85, 86). It has been shown that mitochondrial glutathione (mGSH) controls the fate of hepatocytes in response to TNFα. Its depletion amplifies the power of TNFα to generate reactive oxygen species, compromising mitochondrial and cellular functions that culminate in cell death (87).

Reactive oxygen species (ROS) and oxygen-derived free radicals are the major source of DNA damage (49, 50). Although most of the damage is repaired, cumulative DNA injury due to ROS may be responsible for spontaneous carcinogenesis. ROS are regarded as having carcinogenic potential and have been associated with tumor promotion. Any disturbance of the balance between ROS and endogenous antioxidants in favor of ROS causes an increase in oxidative stress and initiates subcellular changes leading to cancer. Oxidative stress plays a detrimental role in a number of pathological conditions, including cancer (22, 23). Resistance of many cells against oxidative stress is associated with high intracellular levels of glutathione (72-74). Also exposure to several physical and chemical agents can enhance the generation of ROS and can deplete the antioxidant defense. Anticancer drug, baicalein, enhances cytotoxicity. This increase in apoptotic cells may be associated with the depletion of glutathione in Hep G2 cells (80).

One of the aldehydes for the glutathione conjugates is base propenal which plays a key role in DNA damage. DNA strand breaks are caused, directly or indirectly, by a variety of DNA-damaging agents, including ionizing irradiation and oxidative metabolism (51-54). These breaks can have serious consequences, including chromosomal aberrations, increased genetic instability, carcinogenesis and cytotoxicity (55). Bleomycin has demonstrated clinical utility against a variety of neoplasms (treatment of head and neck cancer, Hodgkin's disease and testicular cancer) (57). Bleomycin-induced DNA damage generates base propenal. Base propenal is also generated by antitumor agents including neocarzinostatin and calicheamicin (58), human fibroblasts (59), oxidants such as chromium and peroxynitrite (60, 61) and ionizing radiation (62). These aldehydes undergo Michael addition with cellular nuleophiles such as glutathione and form glutathione conjugates which have been suggested to be responsible for the cytotoxicity of bleomycin and related antibiotics. Significantly, base propenals form pyrimidopurinone (M₁G) adducts with DNA, which as a class represent one of the most abundant background DNA lesions. High levels of M₁G adducts have been found in healthy animals and humans and these adducts have been suggested to be responsible for spontaneous carcinogenesis (21).

Overall, AR and its non-sugar related substrates such as glutathione conjugates play key roles in cellular functions other than glucose metabolism. Therefore, it appears desirable to understand the interaction between AR and substrates including glutathione, aldehyde, and glutathione conjugates which are not sugar family members, to design and screen compounds that may efficiently inhibit or enhance AR's function in catalyzing the reduction of its substrates, and to use the compounds to treat conditions associated with AR or its substrates, for example, drug-resistant tumor cells.

### SUMMARY OF THE INVENTION

The present invention relates to 1) findings in molecular modeling revealing that glutathione conjugate (FIG. 19), a substrate to aldose reductase (AR), can bind to aldose reductase in two distinct orientations (FIG. 1), 2) findings that glutathione conjugate is efficiently reduced by AR (FIG. 17), and findings that fibrates (e.g., bezafibrate) are AR inhibitors (FIGS. 25 & 26). In orientation 1, γ-Glu1 of the conjugate interacts with Trp20, Lys21 and Val47 of aldose reductase (AR), and Gly3 of the conjugate interacts with Ser302 and Leu301 of AR. In orientation 2, the molecule is inverted with γ-Glu1 of the conjugate interacting with Ser302 and Leu301 of AR.

One aspect of the present invention is directed to the design and synthesis of a set of analogs to glutathione conjugate. The analogs are modifications to glutathione conjugate which include substitution and functional group interchange on the glutathione moiety of the glutathione conjugate (FIG. 20), substitutions on the aldehyde moiety of the glutathione conjugate (FIG. 21), variations in the methylene (CH₂) spacer length of the aldehyde moiety (FIG. 20), spacer length variations on the main chain of the glutathione moiety (FIG. 22), and chirality modifications (FIG. 23).

Another aspect of the present invention is directed to the screening and testing of the synthesized analogs to glutathione conjugate by measuring their effect on the activity of AR and identify compounds that are either an AR-inhibitor (AR antagonist) or an AR enhancer (AR agonist).

Another aspect of the present invention is directed to inhibiting AR activity using fibrates. Fibrates include clofibric acid, ciprofibrate, gemfibrizil, bezafibrate, fenofibrate and their analogs.

Another aspect of the present invention is directed to the use of AR-inhibitors or enhancer identified in the treatment of diseases including cancer or the treatment of neoplasm or neoplastic cells. In a preferred embodiment of the present invention, the treatment comprises a step of administering a subject with a disease or a neoplasm a fibrate and a commonly known chemotherapeutics.

### BRIEF DESCRIPTION OF THE DRAWINGS

FIG. 1 shows glutathione propenal in two binding pockets of AR (see also reference 20).

FIG. 2 shows purification of base propenals from DNA.

FIG. 3 shows identification of base propenals from DNA.

FIG. 4 shows formation of the Michael adduct between adenine propenal and reduced glutathione.

FIG. 5 shows ESI⁺/MS of recombinant human AR.

FIG. 6 shows formation of base propenal and glutathionyl base propanol by AR.

FIG. 7 shows cellular metabolism of base propenal.

FIG. 8 shows electrospray mass spectrum of the metabolites of adenine propenal generated in (A) isolated cardiac myocytes and (B) COS-7 cells.

FIG. 9 shows inhibition of AR prevents reduction of the glutathione conjugate of adenine propenal.

FIG. 10 shows transient transfection of COS-7 cells with AR cDNA.

FIG. 11 shows upregulation of AR enhances reduction of adenine propenal.

FIG. 12 shows subcellular localization of AR.

FIG. 13 shows inhibition of AR exacerbates the cytotoxicity of base propenals.

FIG. 14 shows expression of AR in different cell lines.

FIG. 15 shows analogs with substitutions and modifications of the glutathione moiety.

FIG. 16 shows analogs with substitutions and modifications of the aldehyde moiety. Other substitutions for R1 and R2 are Adenine, Guanine, Cytosine, Uracil and Thymine.

FIG. 17 shows formation of glutathione base propenal conjugate (Step 1) and the catalysis by AR (Step 2).

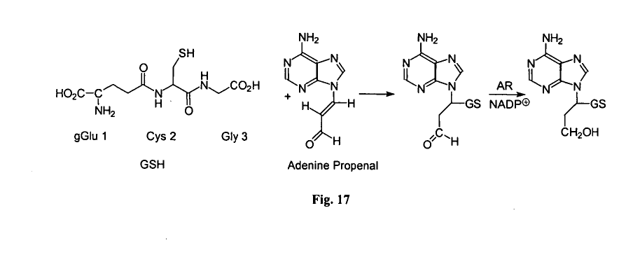

FIG. 18 shows synthesis of glutathione aldehyde conjugates 16-20 set forth in Example 18.

FIG. 19 shows glutathione conjugate.

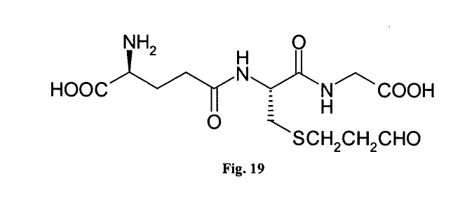

FIG. 20 shows analogs of glutathione acrolein conjugate, wherein R1 is COOH, CONH₂, CH₂OH, COCl, COBr, CH₃, CH₂F, CF₃, H, F, Cl, Br, I, OH, Phosphate, Phosphothioate, SH, SO₂H, SO₃H, NH₂, CN, NO₂, or SR₁ where R₁ is alkyl/aryl; R2 is NH₂, OH, F, Br, Cl, I, SH, Alkyl, Aryl, CN, NO₂, NHR₂ where R₂ is alkyl/aryl, or NR₃ where R₃ is alkyl1alkyl₂/aryl1aryl₂; X-R3 is NH, NR₄ where R₄ is alkyl/aryl, S, O, Se, (CH₂)n; Y is CH₂, O, S, SH, OH, NR₅ where R₅ is H/alkyl/aryl; Z-R4 is O, S, Se, NR₆ where R₆ is H/alkyl/aryl, (CH₂)n; R5 is Alkyl, Aryl, NO₂, CN, F, Cl, Br, I, Phosphate, Phosphothioate, COOH, CH₂OH, CONR₇ where R₇ is H/alkyl/aryl; W is O, S, NR₈ where R₈ is H/alkyl/aryl, CH₂, CHOH; V is S, O, CH₂, Se, SO₃H, CH₂—C₆H₄NO₂; and n is an integer including zero.

FIG. 21 shows analogs of aldehyde substitution, wherein R1 is Ph, 2-furyl, 4-pyridyl, C₅H₁₁—C*H—(OH), F, Cl, Br, or I and R2 is Ph, 2-furyl, 4-pyridyl, F, Cl, Br, or I. The aldehyde in the conjugate can also be substituted by the isomers of 4-hydroxy-trans-2-nonenal (HNE), acrolein and α,β-unsaturated aldehydes, propanal, butanal, pentanal, hexanal, heptanal, ocatanal, nonanal, decanal, acrolein, crotonaldehyde, trans-2-pentenal, trans-2-hexenal, trans-2-heptenal, trans-2-octenal, trans-2-nonenal, 4-hydroxy trans-2-pentenal, 4-hydroxy trans-2-hexenal, 4-hydroxy trans-2-octenal, 4-hydroxy trans-2-nonenal, 4-hydroxy trans-2-decanal, trans, trans-2,4-hexadienal, trans, trans-2,4-heptadienal, trans, trans-2,4-nonadienal, trans, trans-2,4-decadienal, trans-4-decenal, cis-4-decenal, trans-2, cis-6-decadienal, adenine propenal, cytosine propenal, guanine propenal, thymine propenal, uridine propenal, uracil, 2-methyl acrolein, 2-ethyl acrolein, 2-butyl acrolein, phenyl acrolein, methyl phenyl acrolein, core aldehyde 1-palmitoyl-2-(5-oxovaleroyl) phosphocholine, cinnamic acid and it redivatives, naphthalene derivatives, quinone and its derivatives.

FIG. 22 shows extended analog based on glutathione. ★ are chiral atoms, ★′ are atoms not chiral in glutathione but can be made chiral in the analogs and ii★ are not chiral in glutathione which can be made chiral if need arises. A, B. D, E, G, J, L, M are (CH₂) or (CHX) or any other groups. The rest are the same as defined in FIG. 20.

FIG. 23 shows chiral atoms are shown with ★ whereas ★′ are atoms not chiral in glutathione but can be made chiral.

FIG. 24 shows the chemical structure of Bezafibrate and clofibric acid.

FIG. 25 shows the reciprocals of reaction rate and substrate concentration in the absence and in the presence of bezafibrate (0.1 to 100 μM) displayed a partial noncompetitive inhibition pattern with respect to reduction of glyceraldehyde by the recombinant AR in the forward direction.

FIG. 26 shows the measurement of IC50 (the concentration of inhibitors which reduce the enzyme activity by 50%), obtained from a graph of % inhibition of AR activity versus the concentration of inhibitor under saturating substrate condition (10 mM DL-glycealdehyde). The IC50 value of the inhibitor was determined to be 3.8 μM.

FIG. 27 shows the chemical structure of ethyl 1-benzyl-3-hydroxy-2(5H)-oxopyrrole-4-carboxylate (EBPC) and N-(6-chloropyridin-3-ylmethyl)-2-nitroiminoimidazolidine (imidacloprid).

FIG. 28 shows the measurement of IC 50 of Doxorubinin. The IC50 value was determined to be 0.2 μM.

FIG. 29 shows the measurement of IC50 of Daunorunicin. The IC50 Value was determined to be 5 μM.

FIG. 30 shows the measurement of IC50 of Idamycin. The IC50 Value was determined to be 5.6 μM.

FIG. 31 shows the measurement of IC50 of Epirubicin. The IC50 Value was determined to be 5.5 μM.

FIG. 32 shows the chemical structure of α-cyano-4-hydroxycinnamic acid.

FIG. 33 shows the Michaelis-Menten kinetic analysis of α-cyano-4-hydroxycinnamic acid on the AR enzymatic activity. The Ki value was determined to be 0.085 μM.

FIG. 34 shows the measurement of IC50 of α-cyano-4-hydroxycinnamic acid. The IC50 value was determined to be 0.08 μM.

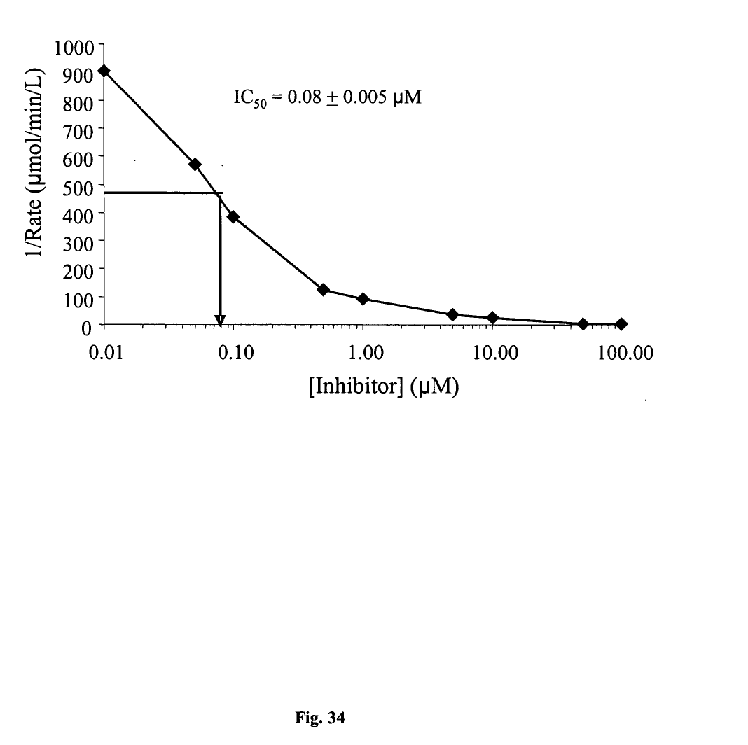

### DETAILED DESCRIPTION OF THE INVENTION

The present invention relates to the findings that aldehyde-glutathione conjugates are reduced efficiently by aldose reductase (AR) with a catalytic efficiency 1000 fold higher than aldehyde as a sole substrate to (AR) (42).

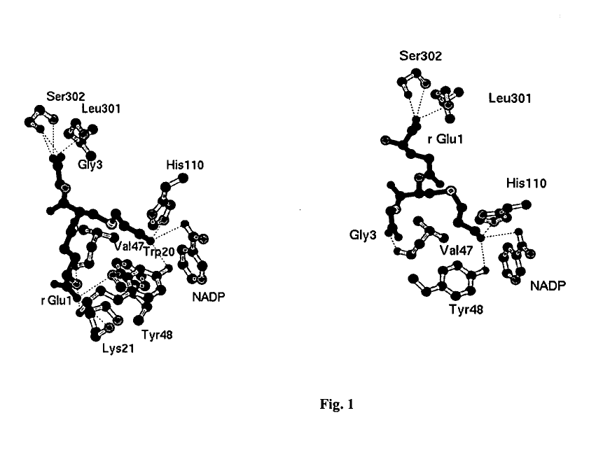

The present invention relates to the molecular modeling and kinetic studies revealing that the glutathione-aldehyde conjugate can bind to two binding orientation in the pocket of AR. In the first binding orientation amino acids residues Trp 20, Lys 21, Val47, Ser302 and Leu 301 of AR (SEQ ID NO: 1) whereas γ-Glu1 of glutathione interacts with a amino acids residues Trp 20, Lys 21 and Val47 and Gly3 of glutathione with Ser302 and Leu301 of the three dimensional structure of AR (SEQ ID NO: 1) as shown in FIG. 1(A). The second binding orientation of AR is defined by the interaction with the amino acid residues of Ser302 and Leu301 wherein γ-Glu1 of glutathione interacts with Ser302 and Leu301 of the three-dimensional structure of AR (SEQ ID NO:1) as shown in FIG. 1(B). It is proposed that the binding conformation of the glutathione conjugates is distinctly different from all the known substrates and inhibitors of AR that are members of sugar metabolism.

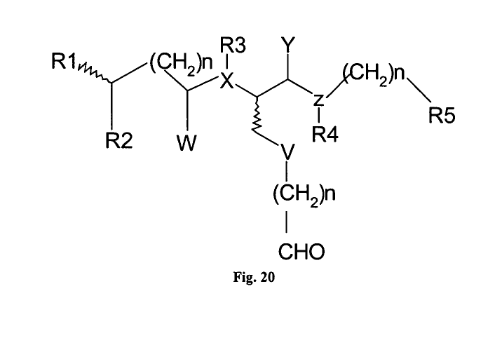

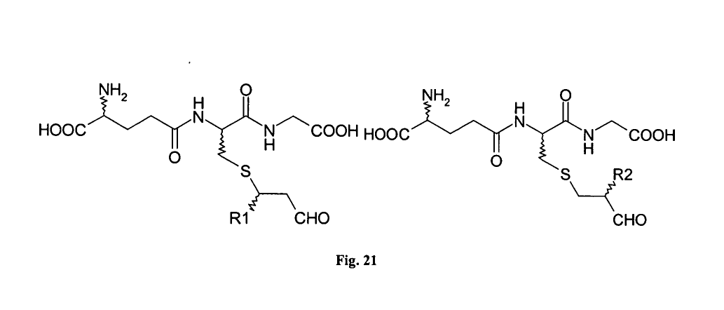

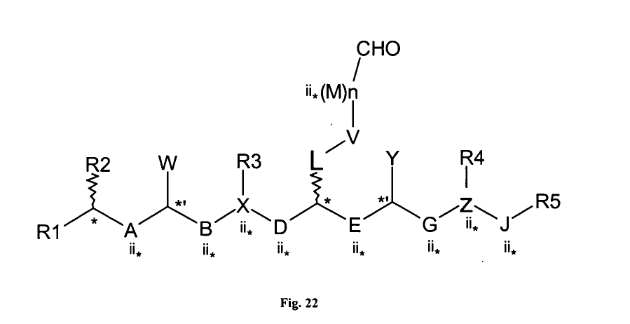

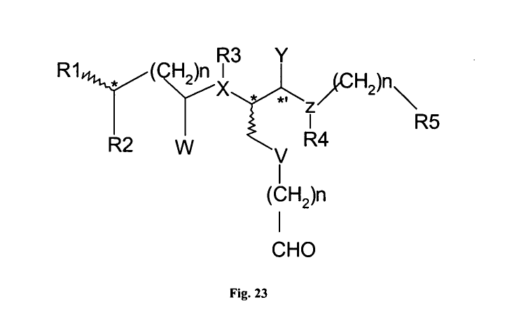

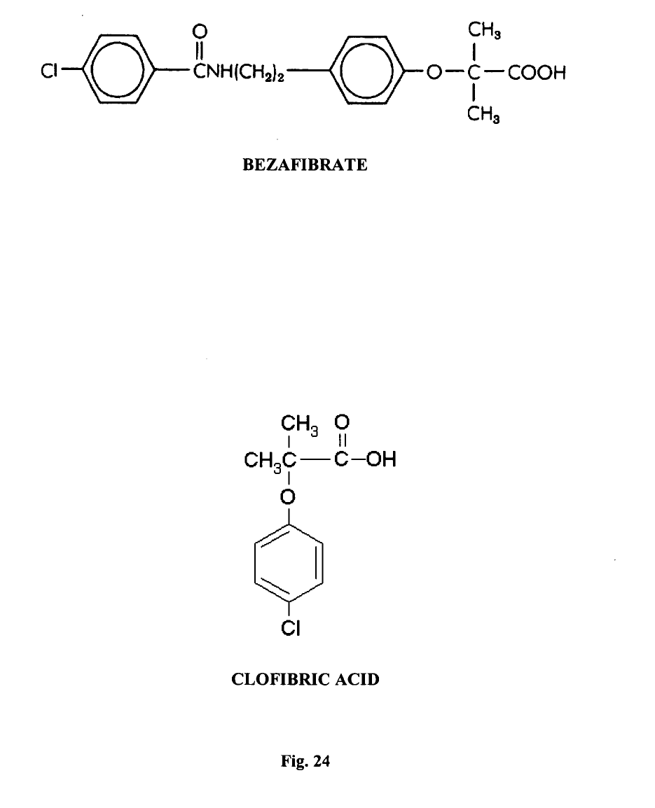

The primary aspect of the present invention is directed to rationally design a set of virtual molecules or analogs to glutathione conjugate that interact with AR as AR ligand which include AR agonist (AR enhancer) that enhance or activate the activity of AR and AR antagonist (AR inhibitor) that inhibit or repress the activity of AR. Methods like QSAR analysis is employed to carry out structure based drug design for AR. The analogs of glutathione conjugates include (1) substitution and functional group interchange on the glutathione moiety (FIG. 20), (2) substitutions on the aldehyde moiety (FIG. 21), (3) variation in the methylene (CH₂) spacer length of the aldehyde moiety (FIG. 20) and (4) spacer length variation on the main chain of the glutathione moiety including different functional groups (FIG. 24) and (5) chirality modifications (FIG. 23). Additionally analogs will be extended to compounds based on the general skeleton shown in FIG. 22 employing different atom types and unsaturation.

Another aspect of the present invention is directed to chemically synthesize a set of analogs to glutathione conjugates. The analogs of glutathione conjugates include (1) substitution and functional group interchange on the glutathione moiety (FIG. 20), (2) substitutions on the aldehyde moiety (FIG. 21), (3) variation in the methylene (CH₂) spacer length of the aldehyde moiety (FIG. 20) and (4) spacer length variation on the main chain of the glutathione moiety including different functional groups (FIG. 22) and (5) chirality modifications (FIG. 23). Additionally analogs will be extended to compounds based on the general skeleton shown in FIG. 22 employing different atom types and unsaturation.

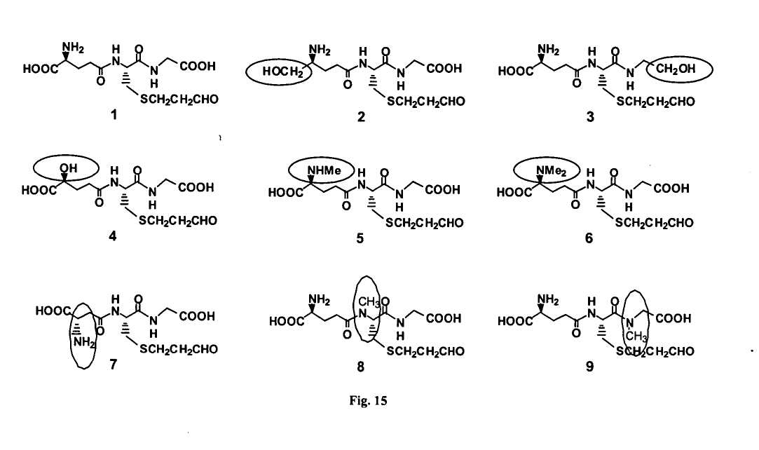

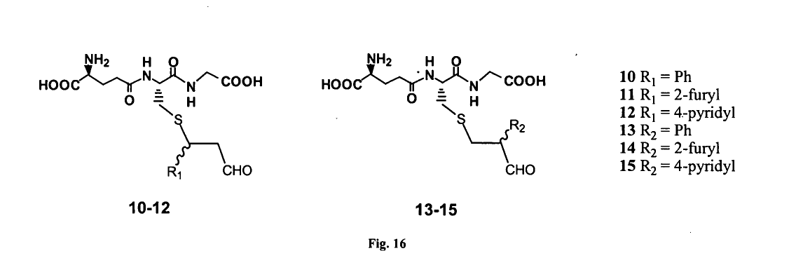

In a preferred embodiment of the present invention, the analogs include glutathione aldehyde conjugates (FIG. 15, compound 1) for systematic structure-activity studies. Analogs to 1 will include (1) substitution and functional group interchange on the glutathione moiety (FIG. 15, compounds 2-9), (2) substitutions on the aldehyde moiety (FIG. 16, compounds 10-15), (3) variation in the methylene spacer length of the aldehyde moiety (Scheme 2, compounds 16-20) and (4) chirality modifications.

The aldehyde moiety of the conjugate can be substituted by isomers of 4-hydroxy-trans-2-nonenal (HNE), acrolein and α,β-unsaturated aldehydes, propanal, butanal, pentanal, hexanal, heptanal, ocatanal, nonanal, decanal, acrolein, crotonaldehyde, trans-2-pentenal, trans-2-hexenal, trans-2-heptenal, trans-2-octenal, trans-2-nonenal, 4-hydroxy trans-2-pentenal, 4-hydroxy trans-2-hexenal, 4-hydroxy trans-2-octenal, 4-hydroxy trans-2-nonenal, 4-hydroxy trans-2-decanal, trans, trans-2,4-hexadienal, trans,trans-2,4-heptadienal, trans,trans-2,4-nonadienal, trans,trans-2,4-decadienal, trans-4-decenal, cis-4-decenal, trans-2, cis-6-decadienal, adenine propenal, cytosine propenal, guanine propenal, thymine propenal, uridine propenal, uracil, 2-methyl acrolein, 2-ethyl acrolein, 2-butyl acrolein, phenyl acrolein, methyl phenyl acrolein, core aldehyde 1-palmitoyl-2-(5-oxovaleroyl) phosphocholine, naphthalene derivatives, quinone and its derivatives.

Another aspect of the present invention is directed to the screening and testing of the compounds or the analogs of glutathione conjugate to determine the cellular and biological activity of the compound in relation to AR. In particular, the compounds are tested as to whether they interact with AR (AR ligands), inhibit (AR-inhibitors or AR antagonists) or enhance (AR-enhancer or AR agonists) the activity of AR. AR-inhibitors are compounds that either compete with substrates in binding to AR or reduce the efficiency of AR in catalyzing glutathione conjugates. AR-enhancers are compounds that in the presence of the enhancer glutathione conjugates/substrates are more efficiently catalyzed by AR.

Another aspect of the present invention is directed to use compounds that are identified as AR ligand in the treatment of conditions where AR is involved or conditions in need of modulating the activity of AR. The conditions are various complications of diseases including cardiovascular disease, diabetes, artheriosclerosis, cancer, cataract, obesity, retinopathy, keratopathy, nephropathy, neurosis, thrombosis, faulty union of corneal injury and neuropathy.

A preferred aspect of the present invention is directed to the role of AR activity and AR-ligand in cancer. It is known that AR activity is increased in cancer cells (68) and glutathione depletion is an important factor in cell death (87). Oxidoreductase plays an important role in cellular metabolism of aldehydes derived from DNA damage caused by, for example, Bleomycin. Glutathione depletion causes cell growth inhibition and enhanced apoptosis in pancreatic cancer cells. In addition, glutathione conjugation has been proposed as an activation mechanism to account for the nephrotoxicity (101). Glutathione conjugation with base propenals will be one of the major pathways to consume glutathione and DNA damage will have significant impact on tumor biology. Therefore, the AR-ligands may provide alternative treatment for cancer. In particular, AR-ligands, e.g., AR enhancers or AR agonists, may cause tumor cells to be more responsive to anti-cancer drug such as Bleomycin or cause tumor cells to be less resistant to anti-cancer drug. Conversely, AR-ligands, e.g., AR inhibitors or AR antagonists, may be developed and identified to minimize the toxicity and side effect to normal cells due to base propenals.

The compounds which are AR ligands, e.g., AR-inhibitors or AR-enhancers, can be administered to a subject in need of modulating AR activity through an administration method or route known to the art. The administration method includes an intravenous administration, an intraperitoneal administration, a subcutaneous administration, an intramuscular administration, an oral administration, a nasal administration, a topical administration, local administration, a transdermal administration, a transmucosal administration, or a pulmonary inhalation.

In one embodiment of the present invention, AR activities are inhibited by fibrates. Fibrates are commonly known to be compounds that decrease serum triglyceride and increase HDL. Fibrates are currently used to improve postprandial triglyceride clearance and reduce the circulating concentration of small, dense LDL. According to methods in the present invention, it is unexpectedly discovered that fibrates are inhibitors of AR. Fibrates include, but are not limited to, ciprofibrate, clofibric acid, gemfibrizil, bezafibrate, and fenofibrate.

Another aspect of the invention is directed to a method of treating neoplasm or neoplastic cells using AR inhibitors. The term neoplasm or neoplastic cells refer to cells that grow in an abnormal way or tissues composed of cells thereof. Normal tissue is growth-limited, i.e., cell reproduction is equal to cell death. Feedback controls limit cell division after a certain number of cells have developed, allowing for tissue repair but not expansion. Neoplastic cells are less responsive to these restraints and can proliferate to the point where they disrupt tissue architecture, distort the flow of nutrients, and otherwise do damage. Neoplasm may be benign or malignant tumors. Benign tumors remain localized as a discrete mass. They may differ appreciably from normal tissue in structure and excessive growth of cells, but are rarely fatal. However, even benign tumors may grow large enough to interfere with normal function. Some benign uterine tumors, which can weigh as much as 50 lb (22.7 kg), displace adjacent organs, causing digestive and reproductive disorders. Benign tumors are usually treated by complete surgical removal. Neoplastic cells of malignant tumors (e.g., cancer) have characteristics that differ from normal cells in other ways beside cell proliferation. For example, they may be deficient in some specialized functions of the tissues where they originate. Malignant cells are invasive, i.e., they infiltrate surrounding normal tissue; later, malignant cells metastasize, i.e., spread via blood and the lymph system to other sites.

Both benign and malignant tumors are classified according to the type of tissue in which they are found. For example, fibromas are neoplasms of fibrous connective tissue, and melanomas are abnormal growths of pigment (melanin) cells. Malignant tumors originating from epithelial tissue, e.g., in skin, bronchi, and stomach, are termed carcinomas. Malignancies of epithelial glandular tissue such as are found in the breast, prostate, and colon, are known as adenocarcinomas. Malignant growths of connective tissue, e.g., muscle, cartilage, lymph tissue, and bone, are called sarcomas. Lymphomas and Leukemias are malignancies arising among the white blood cells.

The role of AR and AR inhibitors. It has been reported that AR and AR inhibitors are involved in the signal trasduction pathway. Studies from several laboratories indicate that inhibition of AR activity by AR inhibitors or AR translation diminishes both the TNFα induced activation of NF-κB and proliferation (95, 119, 71). This inhibition of NF-κB may be related to abrogation of protein kinase C (PKC) signaling and that AR-catalyzed reaction products may be an obligatory requirement for the activation of PKC. Hyper-osmotic stress induces transcription of the AR gene (88), resulting in increased AR mRNA levels (89), followed by rise in AR protein synthesis rate (90) and ultimately increased sorbitol accumulation (91).

Glutathione, in mammalian cells, plays a key role in determining the sensitivity of cells to radiation and drug-induced cytotoxicity (81, 82, 74). Elevation of intracellular glutathione levels is associated with mitogenic stimulation (83), regulation of DNA synthesis (84), control of tumor-cell proliferation by regulating protein kinase C activity (95, 119) and intracellular pH (pHᵢ) (85). The onset of severe tumor-related weight loss (cachexia) in the host is accompanied by a decrease in the rate of cancer cell proliferation and a decrease in glutathione in the tumor (85, 86). It has been shown that mitochondrial glutathione (mGSH) controls the fate of hepatocytes in response to TNFα. Its depletion amplifies the power of TNFα to generate ROS, compromising mitochondrial and cellular functions that culminate in cell death (87). Reactive oxygen species (ROS) and oxygen-derived free radicals are the major source of DNA damage (49, 50). Although most of the damage is repaired, cumulative DNA injury due to ROS may be responsible for spontaneous carcinogenesis. ROS are regarded as having carcinogenic potential and have been associated with tumor promotion. Any disturbance of the balance between ROS and endogenous antioxidants in favor of ROS causes an increase in oxidative stress and initiates subcellular changes leading to cancer. Oxidative stress plays a detrimental role in a number of pathological conditions, including cancer (22, 23). Resistance of many cells against oxidative stress is associated with high intracellular levels of glutathione (72-74). Also exposure to several physical and chemical agents can enhance the generation of ROS and can deplete the antioxidant defense. Hence there is a need for better and more potent compounds to boost antioxidant defense. Anticancer drug, baicalein enhances cytotoxicity. This increase in apoptotic cells may be associated with the depletion of glutathione in Hep G2 cells (80).

AR and AR inhibitor are also involved in diseases such as viral hepatitis or hepatocellular carcinomas. Hepatocellular carcinoma (HCC) is the leading malignancy with a poor prognosis in areas of high hepatitis B and C prevalence (131, 132, 133). After curative resections of HCC, a large proportion of patients develop tumor recurrence within the first 3 years. How to detect these disseminated cancer cells in the perioperative period is a problem. The isolation and identification of tumor cells in a small blood sample by conventional methods is very difficult because the number of malignant cells in the circulation may be extremely small (127; 128; 129; 130). Victims of HCV are at risk for chronic hepatitis, cirrhosis, and hepatocellular carcinoma (Alter et al. 1994; Tang et al., 2004). Takahashi et al., examined age-related changes in the protein and the mRNA expression of aldose reductase in livers of Long-Evans with a cinnamon-like color (LEC) rats, which develop hereditary hepatitis and hepatoma with aging. The levels of the protein and mRNA of aldose reductase increased after 20 weeks, at the stage of acute hepatitis, and were maintained at 60 weeks of age. These results indicated that elevation of aldose reductase accompanied hepatocarcinogenesis and may be related to the acquisition of immortality of the cancer cells through detoxifying cytotoxic aldehyde compounds. (Takahashi et al., 1996). Therefore, further improvement of long-term survival may depend on prevention and treatment of the recurrent tumor. HCC treatment is invasive novel molecular level approach based on the structure fuinction findings, and multidisciplinary interventions might also be important for HCC (Zhou 2002).

In addition, AR and AR inhibitors are involved in the treatment of cancer. Elevated expression of aldose reductase was observed in cancerous lesions of 3′-methyl-4-dimethyl-aminoazobenzene (3′-Me-DAB)-induced hepatocarcinomas. The viability of hepatoma cells in the presence of 3-deoxyglucosone and glyceraldehydes was decreased by an aldose reductase inhibitor, ONO-2235 (5-[1Z,2E)-2-methyl-3-phenylpropenylidene]-4-oxo-2-thioxo-3-thiazolidineacetic acid). Taken together, induction of aldose reductase gene expression during hepatocarcinogenesis may render cancer cells resistant to various toxic carbonyl compounds produced during metabolism or administered as anti-cancer drugs (136).

Cancer cachexia is a most common complication of malignant disease and is a serious clinical problem. Cachexia is characterized by anorexia severe weight loss and progressive tissue wasting (138; 139; 140). The presence of cahexia symptoms deteriorates the effectiveness of cancer therapy or quality of life in patients. In fact a lower frequency of response to anti-cancer therapy and a shorter survival period are not uncommon in patients with cachexia, compared to those without weight loss (139, 140). Cachexia is a complex syndrome, and the cause of cachexia induction is extremely multiple. Tissue wasting, mainly caused by the depletion of skeletal muscle and adipose tissue, is due to metabolic alterations in the host, associated with the enhanced energy requirements for tumor growth (Lazo 1985; Legaspi et al., 1987; Mulligan et al., 1992). Cachexia is a common complication of malignancy and is found in more than 60% of patients with neoplastic disease (1391 140). The effectiveness of cancer therapy depends on the presence or absence of cachexia. Therefore, treatment of cancer cachexia is essential in anti-cancer therapy, because it is expected that inhibition of cachexia, symptoms will result in a longer life span for the patient as well as an improvement of the quality of life.

Recent study has demonstrated that B16 melanoma-induced cachexia in mice is inhibited by ponalrestat, an aldose reductase inhibitor, which has the ability to activate lipoprotein lipase (LPL) activity both in vitro and in vivo. In the study by Kawamura (144-147), the effect of bezafibrate and NO-1886, LPL activators, on B16 melanoma-induced cachectic symptoms was investigated in mice. Treatment with bezafibrate resulted in an attenuation of the decrease in the weight of epididymal fat and whole body lipid observed in mice following intraperitoneal inoculation of B16. The increase in the levels of triglyceride and non-esterified fatty acid, and a decrease in the level of glucose in the blood, which was induced by the presence of tumor, were also restored to that of normal mice after treatment with bezafibrate. The reduction in the weight of epididymal fat and whole body lipid induced by B16 was also ameliorated by NO-1886. Overall, this study demonstrated that cachexia induced by B16 melanoma in mice was alleviated by the LPL activators bezafibrate and NO-1886, suggesting the involvement of the impaired LPL activity in the establishment of cachexia syndrome in mice bearing B16 melanoma (144; 145). Findings propose that ponalrestat, an aldose reductase inhibitor, has a therapeutic potential for the treatment of cancer cachexia. Furthermore ARI were used in nude mice bearing human melanomas G361 and SEKI as well (146).

The effect of ponalrestat on murine adenocarcinoma colon26-induced cachexia was investigated in mice. Mice bearing colon26 subcutaneously lost weight and became cachectic, associated with the tumor growth. Although tumor growth was slightly stimulated when tumor bearing mice were treated with ponalrestat: nevertheless, the drug attenuated the reduction in the weight of body mass, epididymal fat, gastrocnemius muscle and carcass induced by colon26, as well as significantly prolonged the survival of the colon26 bearing mice. Ponalrestat inhibited the production of interleukin-1 (IL-1) from human monocytes stimulated by Lipopolysaccharide (LPS) in vitro, and also suppressed LPS-induced increase of IL-1 in the blood in mice. Overall, this study showed that ponalrestat suppresses IL-1 production both in vitro and in vivo, and inhibits the cachectic symptoms induced by colon26 adenocarcinoma in mice, suggesting that ponalrestat has a therapeutic potential for the treatment of cancer cachexia. (147.)

Changes in glucose metabolism during diabetes are linked to an increased risk for the development of cancer. Increased activity of aldose reductase, the rate-limiting polyol pathway enzyme that converts glucose into sorbitol, mediates pathologies associated with diabetes and is thought to be involved in increased resistance to chemotherapeutic drugs. Thus, increased intracellular sorbitol levels may serve a protective function in cancer cells. These studies by Lee et al., (131) determined whether an inhibitor of aldose reductase could enhance the effectiveness of anticancer agents. Furthermore findings by other groups indicate that treatment with the aldose reductase inhibitor, ethyl 1-benzyl-3-hydroxy-2(5H)-oxopyrrole-4-carboxylate (EBPC), enhances the cytotoxic effects of the anticancer agents doxorubicin and cisplatin in HeLa cervical carcinoma cells. Interestingly, treatment with EBPC in combination with the chemotherapeutic drugs increased extracellular signal-regulated kinase (ERK) activity as compared to treatment with the chemotherapeutic drugs, suggesting a possible role for the ERK pathway in mediating doxorubicin- or cisplatin-induced cell death. Consistent with this possibility, inhibition of ERK activation by the MEK inhibitor, U0126, reversed the EBPC-mediated enhancement of cell death. In summary, these data provide evidence that adjuvant therapy with aldose reductase inhibitors improves the effectiveness of chemotherapeutic drugs, possibly through an ERK pathway-mediated mechanism. (131.) Preclinical report by Shapiro; Many cancer cells contain elevated levels of aldose reductase, indicating that this protein may be involved in cancer cell growth and survival (131).

In adjuvant therapy ARI improves the effectiveness of chemotherapeutic drugs (131). Since HeLa cells express high levels of AR (148) these cells were used to test whether the presence of an ARI could enhance the cytotoxic effects of anticancer agents. Cells were exposed to suboptimum doses of doxorubicin or cisplatin that typically cause minimal cell death in the presence or absence of the ARI, EBPC. Cells treated for 24 h with 0.25 g/ml doxorubicin or 30 μM cisplatin in the presence of 30 or 50 μM of EBPC showed a significant increase in the percentage of dead cells as compared to untreated control. EBPC also enhanced the cytotoxic effects of doxorubicin and cisplatin on cell proliferation. Treatment with EBPC alone at these concentrations had no effect on cell death or proliferation. These data suggest that inhibition of aldose reductase activity increases the effectiveness of doxorubicin and cisplatin in promoting cytotoxic effects in HeLa cells.

HepG2 cells, a stable line of liver cells, were induced to overexpress AR by hypertonicity. Cells that were cultured in hypertonic medium became more resistant to daunorubicin, suggesting that overexpression of AR made the cells more resistant to this drug. This is confirmed by the fact that addition of AR inhibitor sensitizes the cells to this drug again. This information may be important for designing new drugs to treat this deadly disease, liver cancers. This is because 29% of human liver cancers overexpressed aldose reductase (AR) and about 54% of them overexpressed an AR-like gene called ARL-1 that has similar enzymatic activities to AR. (131.)

A new ovarian adenocarcinoma line CABA I cells were characterized by high levels of sorbitol (39±11 nmol/10⁶ cells). Regarding tumor cells, an elevated concentration of sorbitol has been found to induce resistance to cis-platinum in human non-small-cell lung cancer cell lines, by modulating the activity of Na⁺, K⁺ ATPase (149). This body of evidence suggests that accumulation of sorbitol in CABA I cells might be an index of increased metabolic flux through the aldose reductase pathway, by which these fast growing cancer cells would likely enhance their capability of self-detoxification, through reduction of aldehydes or other similar (either endogenous or exogenous) compounds, including anti-cancer drugs. Ovarian carcinomas represent a major form of gynecological malignancies, whose treatment consists mainly of surgery and chemotherapy. Besides the difficulty of prognosis, therapy of ovarian carcinomas has reached scarce improvement, as a consequence of lack of efficacy and development of drug-resistance. The need of different biochemical and functional parameters has grown, in order to obtain a larger view on processes of biological and clinical significance. Biochemical and biological functions suggest in human ovarian carcinoma cells aldose reductase play a significant role especially in relation to their cell detoxification mechanisms during tumor progression (150).

Human aldose reductase-like protein-1 (hARLP-1) was the most prominent tumor-associated AKR member detected in HCC by 2-dimensional electrophoresis (2-DE) and identified by mass spectrometric fingerprinting. The enzyme was found in 4 distinct forms (hARLP-1, 36/7.4 (kd/pI); hARLP-2, 36/7.2; hARLP-3, 36/6.4; and hARLP-4, 33/7.35). In addition, a human aldose reductase-like protein (hARLP-5, 36/7.6) was identified that differed from hARLP-1 by 1 amino acid (D313N), indicating 2 allelic forms of the human aldose reductase-like gene (151). Of these HCC samples, 95% were positive for hARLPs as proven by 2-DE analysis and/or by use of the antibody directed against hARLP. Thus, hARLP is a strong candidate for use as an immunohistochemical diagnostic marker of human HCC.

Induction of AR may, therefore, be a consequence of an adaptive response of cancer cells to the activated metabolism, and may detoxify cytotoxic carbonyl compounds. Moreover, some anti-cancer drugs, such as adriamycin, are known to have an aldehyde group as their functional site. Cancer cells with elevated levels of AR, therefore, may be more resistant to such drugs than cells with lower AR activity. Thus, it is helpful for chemotherapy of a certain cancer in conjunction with AR inhibitors.

The following examples are offered by way of illustration and are not intended to limit the invention in any way. All the references cited in this application are incorporated by reference in their entirety.

### EXAMPLE 1

Synthesis and Analysis of Base Propenals

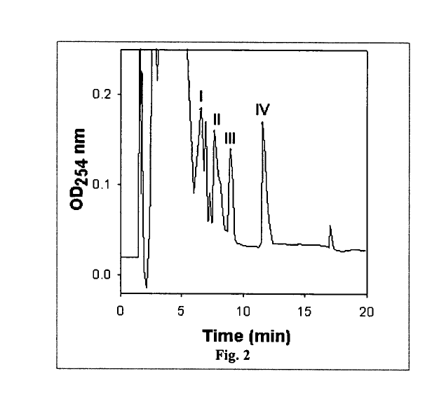

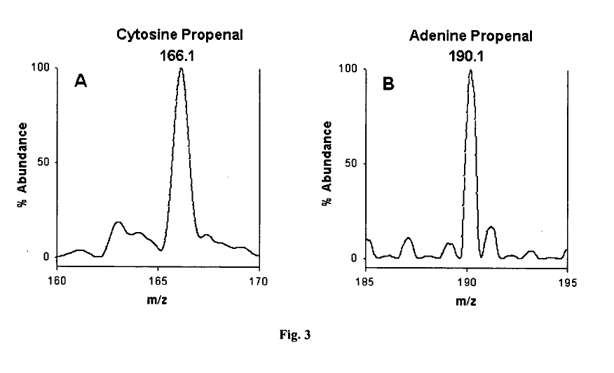

Base propenals generated during Bleomycin (BLM)-induced degradation of calf-thymus DNA have been separated and purified following procedures described earlier (43). Briefly, to generate high concentrations of base propenals, 1.5 mg calf-thymus DNA was incubated with 1 mM Bleomycin A2 and 1 mM Fe(NH₄)₂(SO₄)₂ at 0° C. for 30 min in 50 mM potassium phosphate, pH 7.2. The non-degraded DNA was removed by gel filtration over a G-10 column, and the presence of base propenals in the eluate was measured by the thiobarbituric-acid-reactive-substances (TBARS) test. The peak(s) containing TBARS were collected, pooled and separated by HPLC on a 0.46×10 cm C₁₈ column using a linear gradient of 0 to 100% methanol. The eluate was monitored at 254 nm using a PDA detector. As shown in FIG. 2, the low molecular weight products of DNA degradation eluted as four separate peaks upon HPLC. Each peak was collected and scanned from 210 to 340 nm and the concentration of the individual propenals was calculated using the following extinction coefficients: thymine, ₃₀₃=26.3; adenine, ₂₅₇=34.3; cytosine, ₃₁₂=28.7; and guanine, ₂₆₆=11.3×10³ M⁻¹ cm⁻¹. In a preliminary experiment, a total of 50 μM TBARS were obtained from 1.5 mg calf thymus DNA, which contained 42% thymine, 27% adenine, 10% guanine and 21% cytosine propenals. In addition to synthesis, adenine propenal is commercially available, and to minimize usage of other propenals it will be used for standardizing and troubleshooting experimental protocols. After HPLC separation, each peak corresponding to the retention times of individual propenals was analyzed by electrospray mass spectrometry (ESI⁺/MS) to authenticate the absorption measurements and to establish purity. The mass of the major ions in peaks I-IV corresponded to cytosine (m/z 166.1), guanine (m/z 181.1), thymine (m/z 206.1) and adenine (m/z 190.1) propenal, respectively. The representative ESI⁺/MS profiles of cytosine and adenine propenals are illustrated in FIG. 3. The individual purified base propenals were collected and tested for their ability to serve as substrates of AR and synthesize glutathione conjugates.

### EXAMPLE 2

Synthesis and Analysis of Glutathione Conjugates.

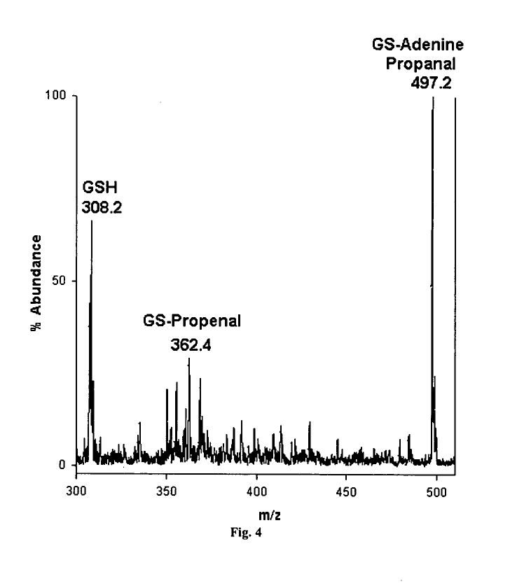

The purified propenals from Example 1 were incubated with a 10-fold molar excess with tritiated reduced glutathione (³H-GSH) in 0.1 M potassium phosphate, pH 7.4. The reaction was monitored spectrophotometerically by following the decrease in absorbance at 312, 327, 303 and 257 nm for cytosine, guanine, thymine and adenine propenals respectively (43). After 30 min of incubation, the radiolabeled glutathionyl conjugates were separated on HPLC and for the case of adenine propenal the conjugate generated was examined by ESI⁺/MS (FIG. 4). The spectrum of the conjugate shows a predominant species with a m/z value of 497.2. Note that the spectrum also shows a molecular ion due to glutathionyl propenal (m/z=362.4), which could have been regenerated from the spontaneous dissociation of the glutathionyl adenine propanal, as has been described before (33). The ion at m/z value of 308.2 appears to be GSH (expected m/z=308) generated perhaps by in-source fragmentation.

### EXAMPLE 3

Production of AR:

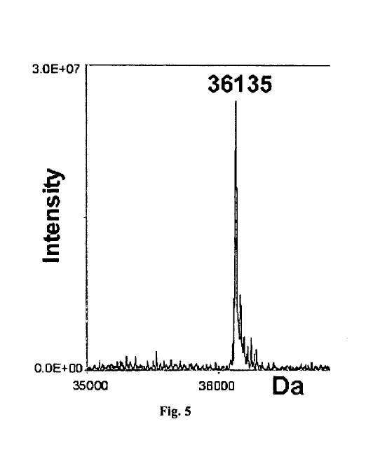

To examine the kinetic properties of AR and its interaction with glutathione conjugate or analogs thereof, the AR over-expressing cells from two laboratories were used. In the case that cells are prepared in accordance reference #45, the expressed protein was purified using a His-Tag affinity column. The eluted protein was collected, reduced and digested by thrombin to partially remove the His-Tag. As shown in FIG. 5, the apparent molecular weight of the protein was 36,135, which is in excellent agreement with the expected molecular weight of 36,134 (AR+his-ser-gly=35,853+281=36,134), indicating that recombinant AR shows no post translational modification. In the case that cells 79) AR was purified following the procedure described in the section on Plan of Attack and its purity determined by SDS gel electrophoresis. No post translational modification was observed upon ESI⁺/MS of AR purified from human tissues. In both the cases the assay was used to follow AR during purification. The human AR has amino acid sequence as shown in SEQ ID NO:1.

### EXAMPLE 4

Reduction of Base Propenals by AR:

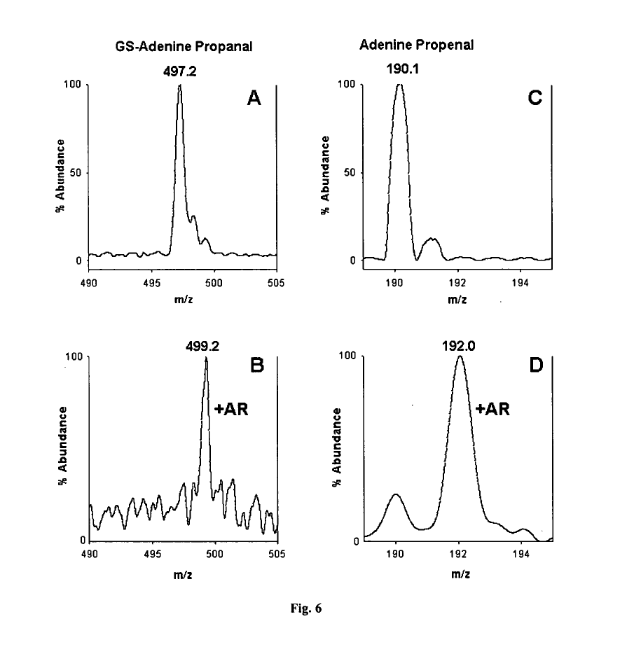

Incubation of AR with NADPH and adenine propenal led to the rapid NADP formation. The glutathione conjugate of adenine propenal was reduced with high efficiency as well. To examine the product of AR-mediated reduction, the reduced propenal as well as its glutathione conjugate were purified by HPLC and examined by ESI⁺/MS. As shown in FIG. 6, reduction of adenine propenal and glutathionyl adenine propanal by AR led to an increase in the m/z value by 2. These data show that AR reduces these aldehydes to their corresponding alcohol. Significantly, no formation of base-free glutathionyl propenol was observed, indicating that reduction prevents the spontaneous release of base from the conjugate. The reduction of adenine propenal and glutathionyl adenine propenal by AR was catalyzed by a 1000- to 10,000-fold high efficiency than glucose or ribose, indicating that propenals are one of the best substrates of AR described so far. AR also catalyzed the reduction of DNA base propenals other than adenine. The glutathione conjugates of these aldehydes were also reduced with efficiency comparable to that of glutathionyl adenine propenal.

### EXAMPLE 5

Cellular Metabolism of Adenine Propenal:

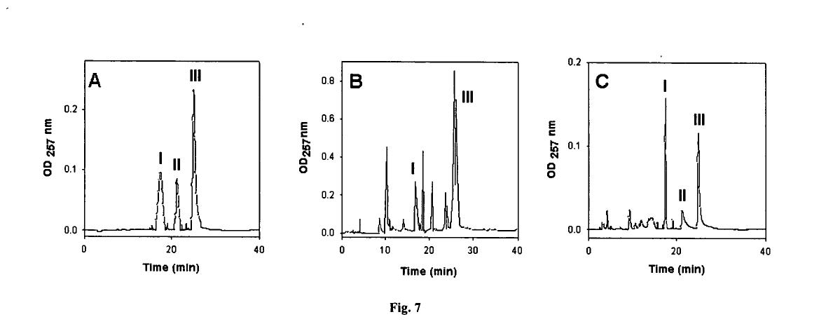

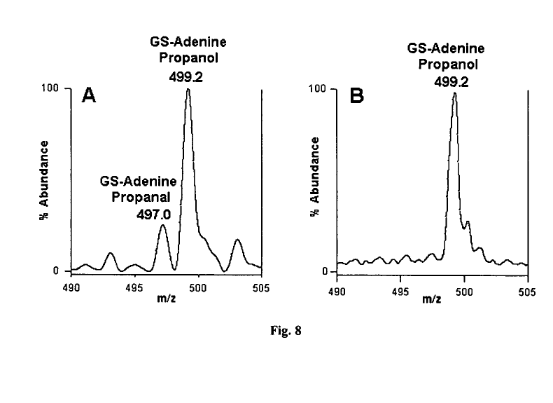

The cellular metabolism of base propenals was examined in isolated rabbit cardiac myocytes and COS-7 cells. The myocytes from rabbit ventricle were isolated as described in (46) and the COS-7 cells were obtained from ATCC. For metabolic studies, the cells were incubated with 10 μM of adenine propenal in Hepes-Ringer's buffer, pH 7.4. After 30 min of incubation, the medium was removed, and the adenine propenal and metabolites in the medium were separated by HPLC. FIG. 7A shows the HPLC separation of reagent adenine propenal from glutathionyl adenine propanal and adenine propenol. The metabolites generated in the medium from cardiac myocytes and COS-7 cells are shown in FIG. 7B and C, respectively. These data show that the glutathione adduct is the major metabolic product generated in these cells, which accounted for approximately 80% of the base propenal consumed. Interestingly, the corresponding acid, which constitutes 50-60% of the metabolites derived from unsaturated aldehydes such as HNE (47), was absent. To establish the structure of the glutathione conjugate peak I of the metabolites was injected into ESI⁺/MS. As shown in FIG. 8, the conjugate formed a strong molecular ion with a m/z value of 499.2, which corresponds to the structure of glutathionyl adenine propanol. No glutathionyl propenal was observed (data not shown). These data show that conjugation of adenine propenal with glutathione is followed by complete reduction of the conjugate to the corresponding alcohol.

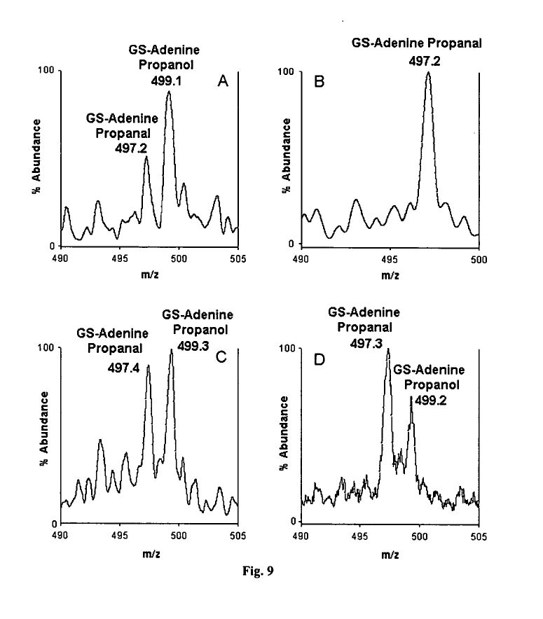

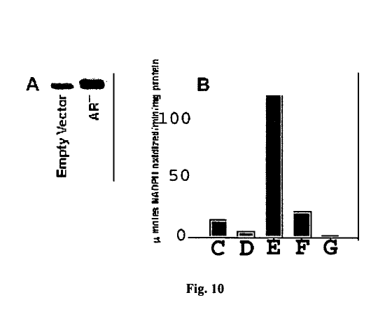

To examine whether the reduction of the conjugate is catalyzed by AR, the metabolism of adenine propenal in cardiac myocytes and COS-7 cells was examined in the presence of two structurally unrelated AR inhibitors, tolrestat and sorbinil. In the presence of these inhibitors the reduction of conjugate was significantly prevented (FIG. 9), such that the predominant species of the conjugate was glutathionyl adenine propanal. To probe the role of AR further, whether upregulation of AR would enhance the reductive metabolism of adenine propenal was examined. To enhance the expression of AR, the COS-7 cells were transfected with AR cDNA using lipofectamine. After 24 h, a 10-fold increase in the expression and a 7-fold increase in the activity of AR were observed as compared to the cells transfected with the empty vector alone (FIG. 10).

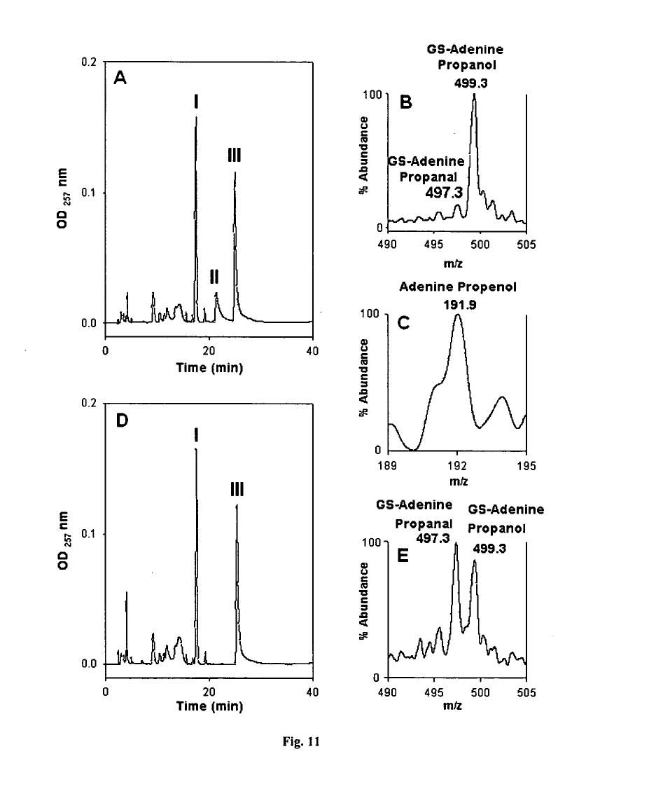

To examine changes in metabolism, the cells transfected with AR cDNA and the empty vector were incubated with 10 μM adenine propenal as described before. After 30 min of incubation, the medium was collected and separated by HPLC. As compared to the vector transfected cells, the AR⁺⁺ cells showed an additional peak with a retention time of 22 min (FIG. 11A), which corresponds to that of adenine propenol, accordingly, adenine propenol was the predominant ion present in this fraction (m/z value of 191.9). The peak was completely abolished when the cells were exposed to adenine propenal in the presence of sorbinil (FIG. 11D). These results suggested that in AR over-expressing cells, a substantial portion of adenine propenal is directly reduced by AR, and that AR-mediated reduction outcompetes for the formation of the glutathione conjugate.

### EXAMPLE 6

Subcellular Localization of AR

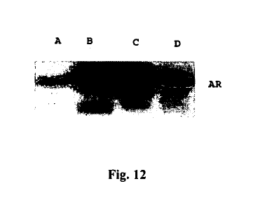

To examine whether AR could reduce aldehydes generated from DNA in the nucleus, the subcellular distribution of AR is determined Subcellular fractionation of pre-B REH cells was performed by differential centrifugation of osmotically swollen cells (48). Centrifugation at 200 g, 10,000 g and 15,000 g were used to separate the nuclei, pallet the mitochondria and membrane, respectively. The nuclear membranes were isolated by centrifugation of the nuclei through a 2 M sucrose cushion at 150,000 g. As shown in FIG. 12, Western blot analysis of SDS-PAGE gels shows that AR is present in high abundance in the cytosol, followed by the light membranes. Significant proportion of the AR protein was also associated with the nuclei. The presence of AR in the nuclei is consistent with the view that it may be involved in the detoxification of DNA-derived aldehydes.

### EXAMPLE 7

Role of AR in the Toxicity of Base Propenals.

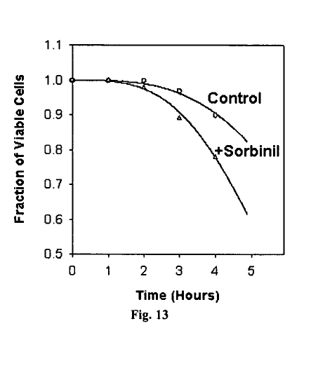

To evaluate the detoxification potential of AR-mediated metabolism, whether the toxicity observed of adenine propenal to COS-7 cells is augmented by AR inhibitors is examined. Incubation of the COS-7 cells with 100 μM adenine propenal led to a progressive loss of cell viability as measured by the MTT assay (FIG. 13), which has a half life of 8.6±0.1 h. However, when the cells were pre-incubated with 100 μM sorbinil, the half life of the cells was significantly decreased to 6.1±0.5 h. These observations imply that reduction of adenine propenal and glutathionyl adenine propenal by AR prevents the cytotoxicity of these aldehydes.

### EXAMPLE 8

Distribution of AR in Different Cells:

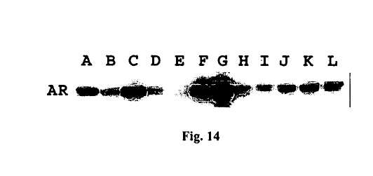

To facilitate studies on the role of AR in propenal metabolism, the abundance of AR protein in several cell lines was examined. These cells were grown in culture under similar conditions. Cell extracts were prepared in protease containing buffer and equal concentrations of cell extracts were loaded on the gel. The AR protein was recognized by anti-AR antibodies. As shown in FIG. 14, the expression of AR in this set of cells was highly variable. AR was most abundant in HeLa (G), H82 (F) and REH (C) cells, while minimal expression of the protein was observed in the K562 (D) and K932 (E) cells. Acceleration of cell death and enhanced cytotoxicity due to exogenously added or endogenously BLM-derived base propenals by AR inhibitors will suggest that AR plays a critical role in the detoxification of base propenals. This conclusion will be further supported by the observations that the K562 cells are more sensitive to BLM/base propenal toxicity and that over-expression of AR in these and COS-7 cells enhances their resistance to base propenaUBLM cytotoxicity. In addition, the sensitivity of HepG2 cells to base propenals will be useful in assessing the contribution of AR in the absence of GSTP1-1-catalyzed glutathiolation. Furthermore, it is expected that the AR inhibitors will restore the sensitivity of the AR-transfected COS-7 and HepG2 cells to the level of the wild type cells, and that the AR inhibitors will not affect the sensitivity of AR-deficient K562 cells. This will help in determining cell type-specific and drug-specific effects. Thus, conditions that lead to enhanced metabolism should be associated with reduced toxicity and vice versa.

### EXAMPLE 9

Rational Molecular Design:

Quantitative structure-activity relationships (QSAR) represent an attempt to correlate structural or property descriptors of compounds with activities. These physicochemical descriptors, which include parameters to account for hydrophobicity, topology, electronic properties and steric effects are determined empirically or, more recently, by computational methods. QSAR studies of many targets have been done in pursuit of rational drug design using activities like chemical measurements and biological assays (125, 126).

### EXAMPLE 10

Building the Model for Glutathione Conjugate Analogs

Compounds that represent the members of the glutathione conjugate analogs are used for the development of 3-D pharmacophores. Analogs of glutathione conjugate will be used in the first round are different (a) substitution on the glutathione moiety, (b) substitutions on aldehyde moiety, (c) length of the aldehyde moiety and (4) chirality (see FIGS. 22-25). The 3D coordinates for the known structures will be obtained from PDB and Cambridge databases. For the compounds without the 3D structures they will be generated in SYBYL on SGI workstations and their energy will be minimized using conjugate gradient procedures employing TRIPOS force field. For the training set their possible conformations will be ascertained where for each analog rotatable bonds will be assigned and a conformational search will be performed allowing the bonds to rotate with a chosen stepwise increment of the dihedral angles. Angle files will be produced and the internal energy corresponding to each valid conformation will be evaluated by molecular mechanics method (options are MM3, AMPAC, Confort).

### EXAMPLE 11.

3-D QSAR Analysis

QSAR with CoMFA provides tools to (1) build statistical and graphical models of activity from molecular structure, (2) uses these models to make accurate predictions for the activity of untested compounds, (3) organizes structures and their associated data into Molecular Spreadsheets, (4) calculates molecular descriptors and (4) performs sophisticated statistical analyses that reveal patterns in structure-activity data. Traditional CoMFA method implemented in SYBYL (104) will be used to perform 3-D QSAR analysis. Currently CoMFA has been widely used to predict biological activity of newly synthesized molecules (104).

### EXAMPLE 12.

Model Validation:

Partial least squares regression will be used to analyze the statistically significant model for the training set by correlating variations in their biological activities with variations in their interaction fields. Using optimal number of components the final partial least squares analysis will be carried out without cross-validation to generate a predictive model with a conventional correlation coefficient. For the training set, two different alignment strategies will be examined using the program FlexS (102, 105). In FlexS physicochemical properties of molecules to be superimposed will be approximated as density distribution in space in terms of associated Gaussian finctions. These functions will be used to automatically superimpose a flexible molecule onto a rigid template molecule (glutathione moiety). For each alignment the interaction field between the ligands and a water probe will be calculated. The variables obtained for each compound will be used to generate the Smart Region Definition/Fractional Factorial Design (SRD/FFD). Subsequently, the SRD procedure will be used to carry out the variable selection on groups of variables chosen according to their positions in 3-D space.

The docking analysis will be performed using a two-stage docking procedure applying the program AutoDock, which has been shown to successfuilly reproduce experimentally observed binding modes (25, 106). The interaction energy of ligand and AR will be evaluated using atom affinity potentials calculated on a grid similar to that described by Goodford (120). In the second step low-energy complexes will be reranked according to the interaction energy calculated with a more detailed energetic model based force field. For this second step, the complexes of the AutoDock energy ranking will be selected. The protein structure will be held fixed during the minimization, whereas the ligand will be allowed to change its conformation and position in the binding pocket. The calculated GRID contour maps will be viewed superimposed on the structures of AR and inspected manually.

### EXAMPLE 13

Screening a Virtual Library of Compounds

The flexible-ligand/grid-potential-receptor docking algorithm (121) will be carried out automatically on Available Chemicals Directory library of 153,000 available chemical compounds (MDL Information System, San Leandro, Calif.). Molecular Design Limited (MDL) Information Systems is a recognized leader in discovery informatics for the life sciences and chemistry in industry and academia. The database contains all the chemical compounds that are commercially available with their complete details such as the vender, solubility and so on. Any hit generated using this approach will be purchased or custom synthesized for further studies. Each compound will be assigned a score according to its fit with AR, which took into account continuum as well as discreet electrostatics, hydrophobicity and entropy parameters. Also subroutines FlexX, Cscore, FlexS, CombiFlex LeapFrog will be used to design potent compounds.

### EXAMPLE 14

Chemical Synthesis of the Modified Glutathione Conjugates

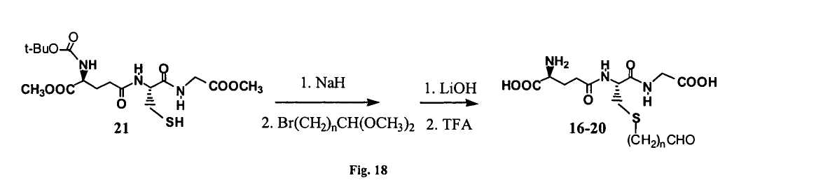

Sets of glutathione aldehyde conjugates (FIG. 15, compound 1) are synthesized for systematic structure-activity studies. Analogs to 1 will include (1) substitution and functional group interchange on the glutathione moiety (FIG. 15, compounds 2-9), (2) substitutions on the aldehyde moiety (FIG. 16, compounds 10-15), (3) variation in the methylene spacer length of the aldehyde moiety (FIG. 18, compounds 16-20) and (4) chirality modifications. Approximately 20 milligrams of each target will be synthesized for initial screening. Larger amounts of the most promising compounds will be synthesized for advanced studies, such as the interaction with AR. The purity of all compounds will be established by HPLC analysis. All the compounds will be characterized by proton and carbon NMR, by high resolution mass spectrometry and by other techniques (optical rotation, elemental analysis, single crystal x-ray analysis) as appropriate.

The set of compounds 2-9 with substitutions and modifications of the glutathione moiety will be prepared by assembly of the modified glutathione from the known component parts (122) via standard solution phase techniques. Compounds 10-15 will be prepared by reaction of glutathione with the appropriate α,β-unsaturated aldehyde (123). Analogs where R1 and R2 are like DNA bases will be chemically synthesized in addition to the biochemical methods. The set of compounds 16-20 will be prepared as illustrated in FIG. 18. Protected glutathione analog 21 can be prepared in five steps from glutathione as described in reference #124. It can be readily alkylated with a series of homologous ω-bromoacetals and the intermediates will be deprotected to give compounds 16-20 where the length of the methylene spacer can be varied. Initially compounds with 3≦n≦7 are prepared. Finally, the chirality and amino acid sequence of the glutathione moiety will be modified. The incorporation of unnatural amino acids as compounds of interest derived from these would presumably resist enzymatic function in vivo. The D-amino acids necessary for this work are commercially available.

### EXAMPLE 15

Biological Activity of AR—Reduction of Base Propenals and their Glutathione Conjugates by AR.

The AR activity will be determined at 37° C. in 100 mM phosphate buffer, pH 7.0 containing 0.15 mM NADPH and the appropriate concentration of the base propenal, by monitoring the rate of disappearance of NADPH at 340 nm. A characteristic feature of AR is its sensitivity to thiol oxidation, which alters its kinetic properties and inhibitor sensitivity. Therefore, all the buffers to which the enzyme is exposed contain thiol-reducing agents such as dithiothreitol (DTT). Because propenals react avidly with thiols, true catalysis of these substrates cannot be measured in the presence of thiols. Therefore, stored AR (which tends to oxidize even in the presence of thiols) will be thoroughly reduced by incubating with 0.1 M DTT at 37° C. for 1 h in 0.1 M Tris-HCl, pH 8.0. This treatment reduces all 7 cysteine residues of the enzyme and minimizes day-to-day variations in the properties of the enzyme. However, reduced AR is rapidly oxidized in air. Therefore, before each experiment, DTT will be removed from the enzyme by gel filtration on a PD-10 column, equilibrated with K-phosphate buffer containing 1.0 mM EDTA. All solutions used for enzyme assay and storage will be saturated with argon. Initially, the k_{cat} and Kₘ values of AR with adenine, guanine, cytosine and thymine propenal are determined. The kinetic parameters of AR will be determined from a complete initial velocity profile at different concentrations of NADPH and adenine propenal, using the following equation for sequential ordered reaction scheme (followed by AR):
v=(Vₘₐₓ·A·B)/(Kᵢₐ·K_{b}+KₐB+K_{b}A+AB),
where A=NADPH and B is adenine propenal. Substrate inhibition, if any, should correspond to the following equations
v=(Vₘₐₓ·A·B)/{Kᵢₐ·K_{b}(1+B/K_{ib}+KₐB(1+K_{ib})+K_{b}A+AB)}
or
v=(Vₘₐₓ·A·B)/Kᵢₐ·K_{b}+KₐB+K_{b}A+AB²/K_{ib}.

Correspondence of the data to the above rate equations will be statistically assessed using well established methods (78). Determination of K_{ib} will be useful in assessing whether at high concentrations of propenals prevent their own detoxification. IC₅₀ values will be calculated from median effect plots following the methods described in (92).

### EXAMPLE 16

Kinetic Data Analysis

Individual saturation curves used to obtain steady-state kinetic parameters will be fitted to a general Michaelis-Menton equation. In all cases, the best fit to the data will be chosen on the basis of the standard error of the fitted parameters and the lowest value of σ, which is defined as the sum of squares of the residuals divided by the degrees of freedom (n−1). For steady-state kinetic analysis, n represents the number of velocity measurements. The substrate concentration will be varied over a range extending from 0.2 to 5-7 times the Kₘ. The initial velocity will be measured at 7-9 different concentrations of each substrate. Multiple (4-6) data sets will be collected for each measurement.

### EXAMPLE 17

Biological activity of AR in Presence of Compounds:

Binding of substrates to the AR family is facilitated by the presence of NADPH. Upon binding, NADPH induces a large conformational change in these proteins, which enhances binding. Moreover, NADPH binding quenches the intrinsic fluorescence of the protein and results in the appearance of an additional emission band at 450 nm. The 450 nm band has been suggested to be due to the formation of a charge-transfer complex between the reduced coenzyme and the tryptophan residues located at the active site (96). The emission of this band is quenched upon substrate binding to AR. This method will be a valuable method to test all the compounds (glutathione conjugates, designed and synthesized) for their binding capability to AR.

Fluorescence spectra will be recorded on a fluorescence spectrophotometer. Excitation wavelength of 290 nm and an emission wavelength of 335 or 345 nm will be used for the fluorometric titrations. Aliquots of the protein will be equilibrated with 2.0 ml of 0.15 M potassium phosphate, pH 7.4. The fluorescence of the protein will be measured before and after the addition of 2-20 μl of the pyridine nucleotides. To minimize nucleotide absorbance, a 5×10-mm cuvette will be used for titration with NAD(H). The protein concentration will be measured by the Bradford dye binding method (76). Fluorescence titration data will be fitted to a binding equation that takes into account the corrections for scatter, dilution and cofactor absorbance (77).

### EXAMPLE 18

Measurement of AR Activity in AR with New Compounds or AR Ligands

The reductase activity will be measured in 250 mM K-phosphate, pH 6.0, containing 0.1 mM NADPH. The substrates will be dissolved in the buffer or in acetonitrile. The final concentration of acetonitrile in the cuvette will be kept below 4%. The catalytic activity will be determined with para-nitrobenzaldehyde (final concentration=400 μM), 9,10-phenanthroqunione (9,10-PQ; 50 μM) and androstane dione (30 μM). Additionally, the catalytic activity of the protein will be determined with 50 mM glucose or 10 mM DL-glyceraldehyde, or 1 mM 4-hydroxy trans-2-nonenal (HNE) and or its glutathione conjugate (GS-HNE). The reference cuvette will contain all the components of the mixture except the substrate. The enzyme activity will be calculated as nmoles of NADPH oxidized/mg protein/min. For determining the reverse activity (alcohol oxidation), alcohols corresponding to the above-mentioned aldehydes will be used with NADP as the cofactor.

### EXAMPLE 19

Cellular Metabolism of Conjugate and New Compounds:

Compounds generated in Example 18 and tested for binding and the kinetic parameters with AR will be tested for their cellular properties following the methods described in the Preliminary Results section A.4. and A.6. In addition other cell lines important for cancer like MCF-7, SKBR-3, MAD-MB-231, T47D, HEP G2, 293 Lincap will be included in this study along with the normal cell lines MCF-10A, MCF-10F and HBL-100.

### EXAMPLE 20

Fibrate as AR Inhibitors

In this experiment, fibrate (e.g., Bezafibrate, See FIG. 24) exhibits a partial noncompetitive inhibition pattern with respect to the reduction of glyceraldehyde by the recombinant hAR in the forward direction. The IC50 value of bezafibrate for human AR was determined to be 3.8 μM. DL Glyceraldehyde, NADPH, and Bezafibrate (2-[4-[2-(4-chlorobenzamiso)ethyl]phenoxy]-2-methyl-propionic acid) were purchased from Sigma-Aldrich. All other chemicals used were of the highest purity available.

Human aldose reductase was recombinantly expressed in E. coli BL21, purified, and used for testing the efficacy of Bezafibrate in regulating aldehyde reduction reaction. E. coli BL21 was grown overnight (16 h) at 37° C. with shaking (250 rpm) in 100 ml of Luria-Bertani (Miller) broth (25 g/l), supplemented with ampicillin (50 μg/ml). Over night grown culture was inoculated (25 ml/l) into four 3 l flasks each containing 11 LB supplemented with ampicillin (50 μg/ml) at 37° C. with shaking (250 rpm) for 3 to 4 h until an attenuance (A₆₀₀) of ˜0.7. Isopropyl β-D-thiogalactoside (IPTG, 1 mM) was added to the culture and was further incubated for 3 h to induce the expression of human aldose reductase gene. Cells were harvested by centrifugation at 10,000 g for 15 min at 4° C. in a Beckman JLA-16250 rotor. Cell pellets were resuspended in 80 ml of Talon extraction/wash buffer, (pH 7.9, Clonetech), and lysed by sonication with ten pulses (30 s each) and centrifuged at 12 000 g. The supernatant was collected and mixed with 5 ml of Talon metal affinity matrix (Clonetech), equilibrated in Talon extraction/washing buffer and incubated for I h at 4° C. to allow the binding of the protein. The slurry was then transferred into a column allowing the matrix to pack and the supernatant to pass through at a flow rate of 0.5 m/min. The column was washed with 50 ml of Talon extraction/wash buffer, and the enzyme was eluted with 50 ml of Talon elution buffer. The eluted protein was dialyzed overnight using Spectra/Por 5-8 kDa MWCO at 4° C. in 50 mM sodium phosphate buffer (pH 7.0) containing 1 mM 2-mercaptoethanol. His-tag from the dialyzed protein was removed using thrombin cleavage kit (Novagen) by adding 1 μl of thrombin to 4 mg of protein and incubating overnight at room temperature. The purity of the protein at each stage of purification was assessed by SDS PAGE, and staining the gels with Coomassie Blue. Protein was quantified by measuring the OD at 280 nm and, one unit of activity corresponds to 1 μmol of coenzyme utilized/min, based on a molar absorption coefficient (ε340) of 6,220 M⁻¹ cm⁻¹

Activity with various concentrations of DL glyceraldehyde in the absence and, in the presence of various concentrations of Bezafibrate was determined by monitoring the change in NADPH concentration at room temperature 28±2° C. in a Beckman DU600 model spectrophotometer by measuring the absorbance at 340 nm, in 0.01 M sodium phosphate buffer (pH 6.2). Kinetic parameters were obtained from initial-rate activity measurements, with substrate concentrations of 0.05 mM to 10 mM. Each individual rate measurement was done in duplicate. At least three independent determinations were performed for each kinetic constant. Values were calculated using Sigmaplot Ver 8.0 (SPSS Inc.) using a non-linear Marquardt's regression algorithm that computes the coefficients (parameters) of the independent variable(s) that give the “best fit” between the equation and the data. Inhibition-constant (Ki) and, the IC₅₀ values for bezofibrate was calculated from the secondary plot of slope values from the double-reciprocal plot versus inhibitor concentration and from the plot of rate of reaction versus inhibitor concentration respectively.

Recombinant Aldose reductase was purified and characterized as a single band (36 kDa) on 10% SDS-PAGE. The purified enzyme exhibited enzyme aldose reduction activity. Flouresence quenching of the purified enzyme was observed as fluorescence emission band of nucleotide free aldose reductase protein and the appearance excitation band upon binding by the NADPH.

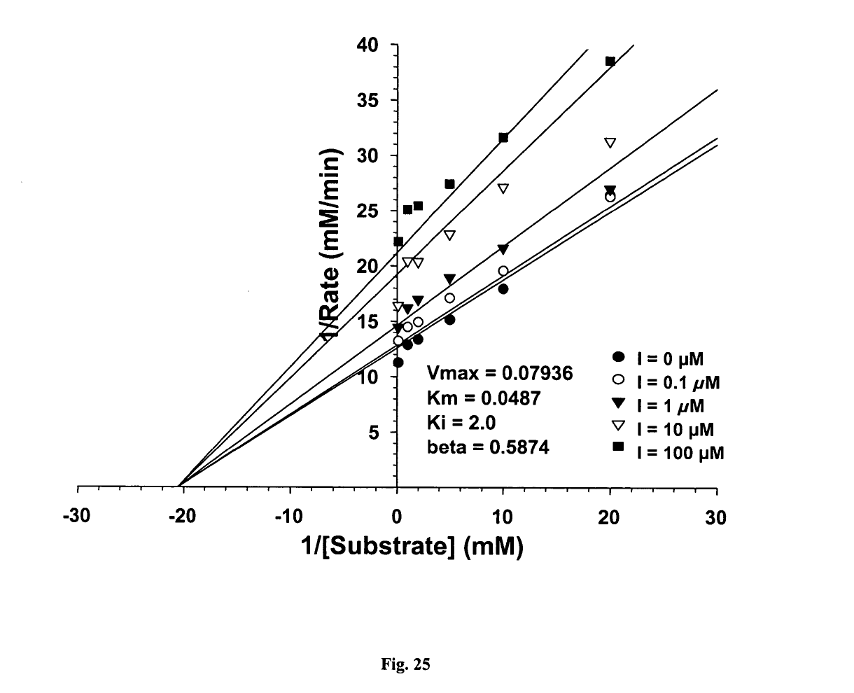

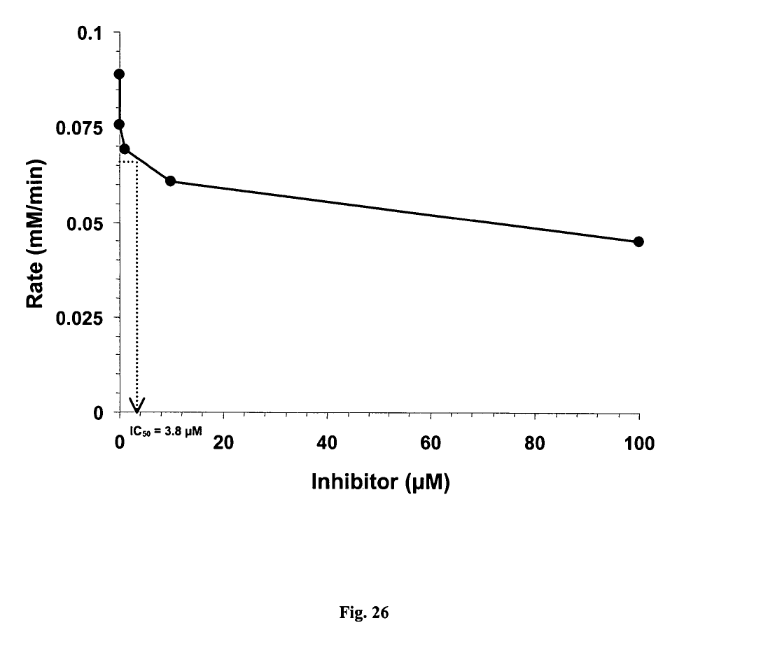

Lineweaver-Burk plot (FIG. 25) of the reciprocals of reaction rate and substrate concentration in the absence and in the presence of bezafibrate (0.1 to 100 μM) displayed a noncompetitive partial inhibition pattern with respect to reduction of glyceraldehyde by the recombinant human aldose reductase in the forward direction. The initial rates in the presence of Bezafibrate were analyzed by using equation 1:
v=Vₘₐₓ/((1+Kₘ/S)*(1+I/Kᵢ)/(1+I*beta/Kᵢ)) (1)
where, v=rate of reaction, Vmax=maximum initial velocity for the uninhibited reaction, Km=Michaelis constant in the absence of inhibitor, Ki=inhibition constant and beta=the rate constant when enzyme substrate complex breaks down to Enzyme and Product. Fitting the data to eq 1 yielded the apparent noncompetitive inhibition constant (Ki=2.0). The IC50 value of the inhibitor was determined to be 3.8 μM (FIG. 26).

Other fibrates, such as gemfibrozil and clofibric acid, have also demonstrated inhibitory effect on AR activity. For example, the Ki value of gemfibrozil is 2.74±0.072 μM and the IC value thereof is 3±0.2 μM. The Ki value of clofibric acid is 1.04±0.047 μM and the IC value thereof is 1.2±0.1 μM (See Table I)

### EXAMPLE 21

Other Compounds as AR Inhibitors

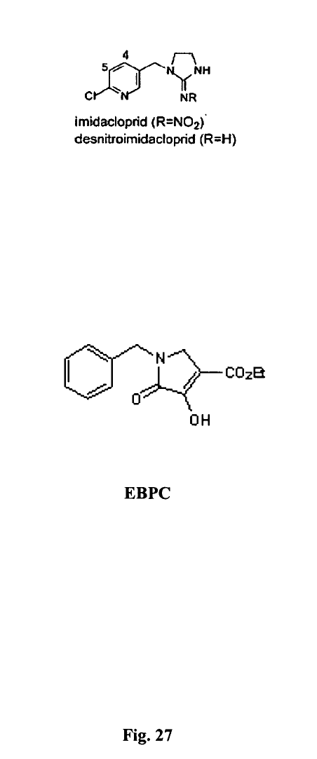

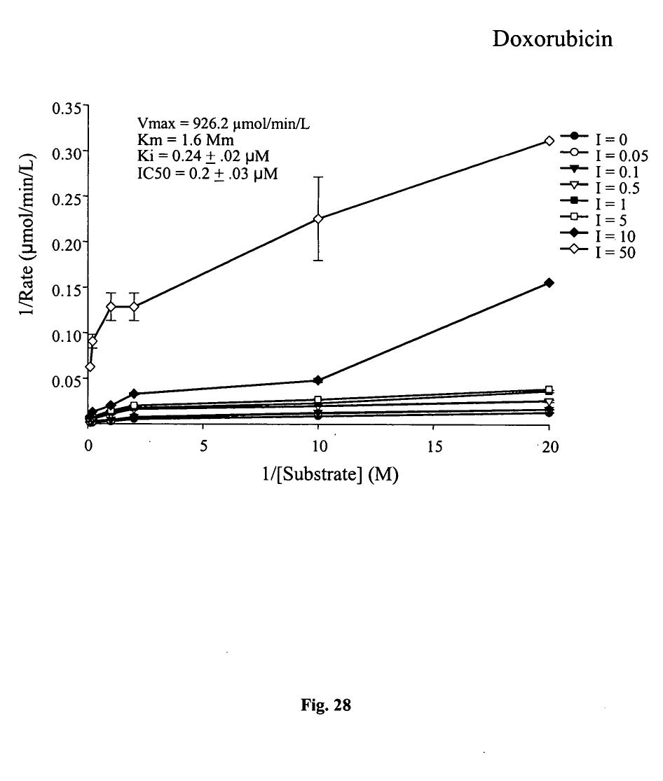

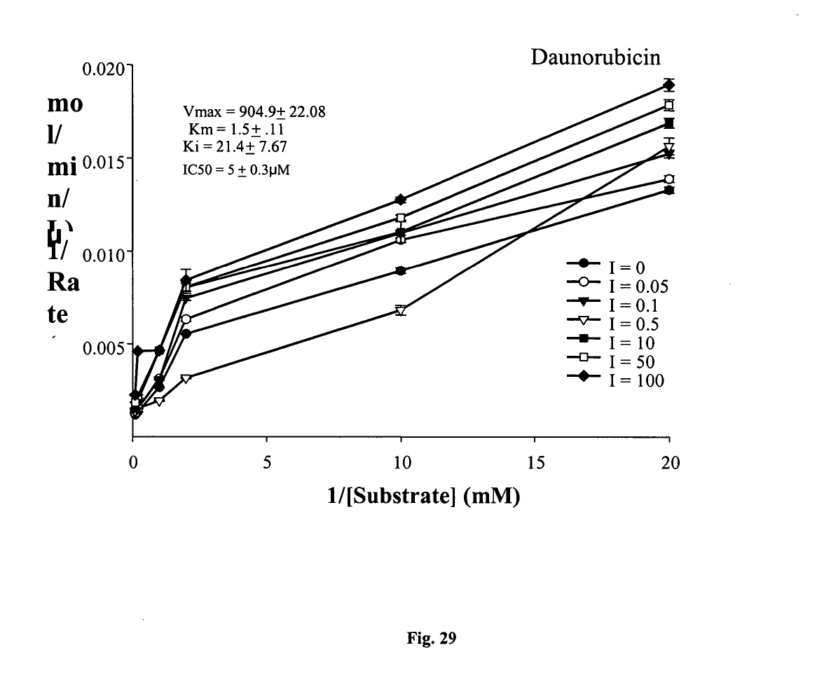

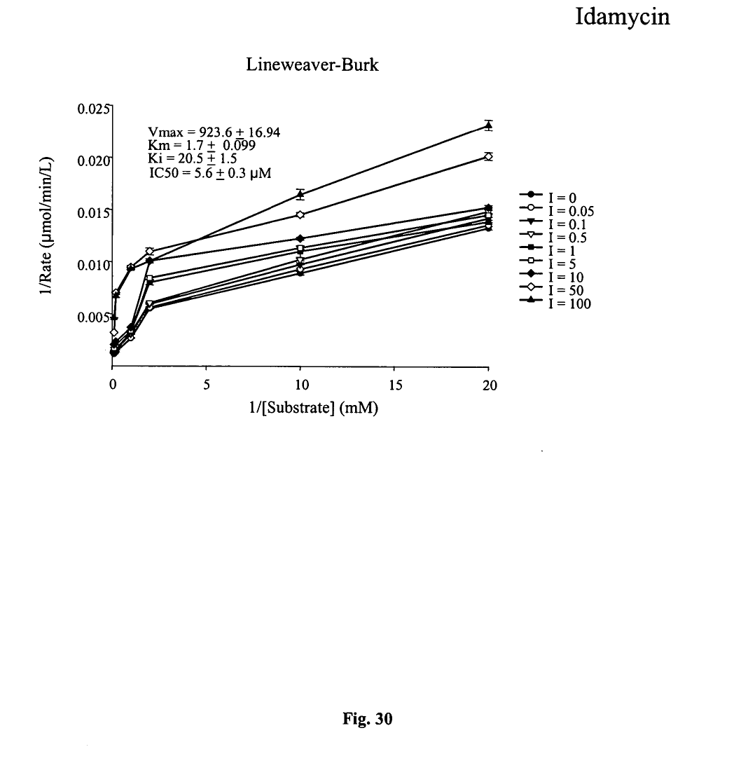

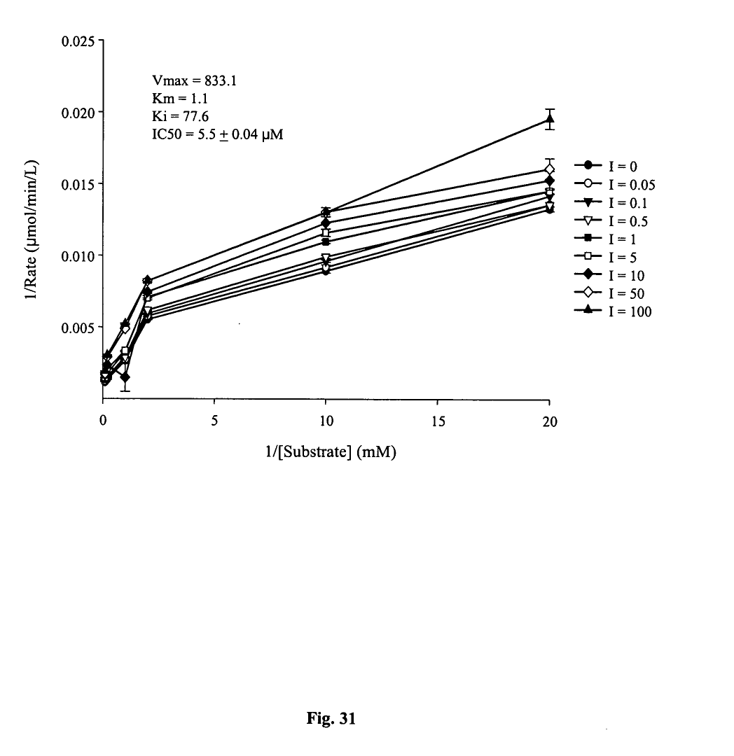

It has been reported that ethyl 1-benzyl-3-hydroxy-2(5H)-oxopyrrole-4-carboxylate (EBPC) inhibits rat aldose reductase (152). In the present application, we find that an analog of EBPC, N-(6-chloropyridin-3-ylmethyl)-2-nitroiminoimidazolidine (Imidacloprid, See FIG. 27 for its chemical structure), is also an AR inhibitor with Ki value of 12.6 μM and IC value of 1.5 μM. In addition, doxorubicin and its analogs are found to have inhibitory effect on AR activity. In experiments similar to what is described in Example 20, anthracyclines (e.g., doxorubicin) exhibits a partial noncompetitive inhibition pattern with respect to the reduction of glyceraldehyde by the recombinant hAR in the forward direction. The IC50 value of doxorubicin for human AR was determined to be 0.2 μM (See FIG. 28). Other compounds, such as daunorubicin (See FIG. 29); idamycin (See FIG. 30); epirubicin (KI value is 77.6, See FIG. 31); sorbinil and zopolrestat also show inhibitory effects on AR activity (See Table I). All the chemicals used were of the highest purity and are available purchased from Sigma-Aldrich and other commercial suppliers.

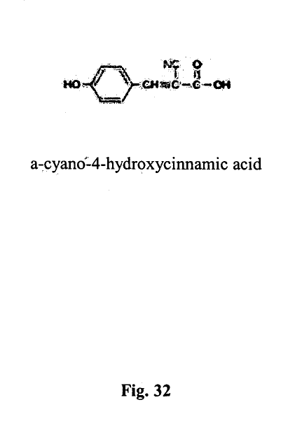

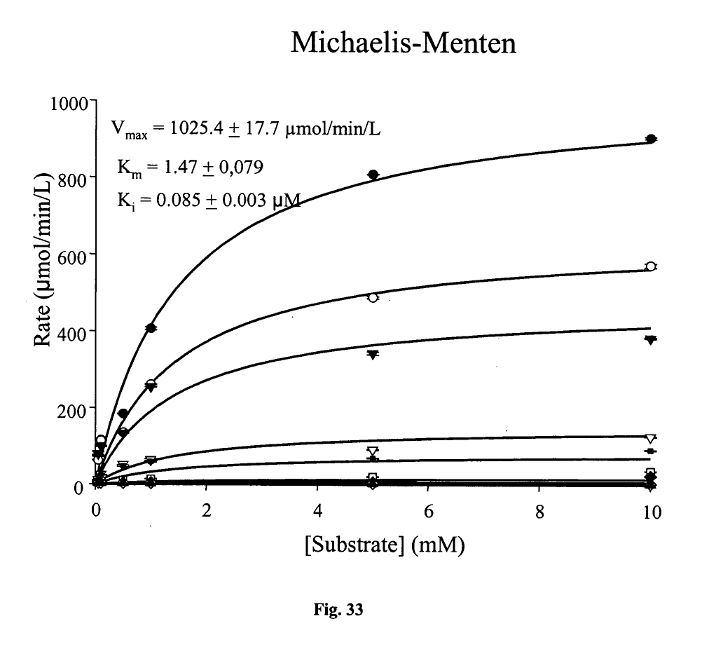

Researchers have evaluated the inhibitory effect of Cinnamomum cassia bark-derived compounds against rat lens aldose reductase (153). It has been found that cinnamaldehyde and quercitrin exhibit high potency in inhibiting rat AR while ainnnamyl alcohol, trans-cinnamic acid and eugenol exhibit only weak inhibition against rat AR. In the experiment similar to Example 20, it is found in the present invention that α-cyano-4-hydroxycinnamic acid, See FIG. 32 for its chemical structure) exhibits a partial noncompetitive inhibition pattern with respect to the reduction of glyceraldehyde by the recombinant hAR in the forward direction. The IC50 value of α-cyano-4-hydroxycinnamic acid for human AR was determined to be IC₅₀=0.08±0.005 μM (Kᵢ=0.085+0.003 μM) (See FIGS. 33 & 34). α-cyano-4-hydroxycinnamic acid used is of the highest purity and purchased from Sigma-Aldrich and other commercial suppliers.

The IC 50 and Ki values of compounds which are inhibitory to AR activity and are measured in the present invention are summarized in Table I.

TABLE I

| Compound | K₁ (μM) | IC₅₀ (μM) |
|---|---|---|
| Bezafibrate | 2.0 | 3.8 |
| Gemfibrozil | 3.5 ± 0.79 | 6.5 ± .02 |
| 2-4-Chlorophenoxy | 1.04 ± 0.047 | 1.2 ± 0.1 |
| clofibric acid | | |
| Sorbinil | 0.4 ± 0.09 | 2.1 ± 0.05 |
| Zopolrestat | 0.04 ± 0.002 | 0.062 ± 0.002 |
| α-cyano-4- | 0.085 ± 0.0003 | 0.08 ± 0.005 |
| hydroxycinnamic acid | | |
| Imidcloprid | 12.6 | 1.5 |
| Doxorubicin | 0.24 ± 0.02 | 0.2 ± 0.03 |
| Idamycin | 20.5 ± 1.5 | 5.6 ± 0.3 |
| Epirubicin | 77.6 | 5.5 ± 0.04 |
| Daunorubicin | 21.4 ± 7.67 | 5 ± 0.3 |

### EXAMPLE 22

Further Experiments

The above experiments in the present invention provide direct and driving rationale for future studies in following directions. a) The role of AR in the reduction and detoxification of other DNA-derived aldehydes. The detoxification of these aldehydes may be essential for preventing the formation of covalent DNA adducts and DNA-DNA or DNA-protein crosslinks, especially by the reactive dicarbonyls. Given the broad substrate specificity of AR and the structural similarity of these aldehydes to other AR substrates, it is likely that a wide range of DNA-derived aldehydes is reduced by AR. Hence, this possibility will be tested. Thus a new role of AR may emerge, which may provide experimental access to the currently unknown metabolic, cytotoxic, and mutagenic effects of DNA-derived aldehydes. b) Transport of the reduced and non-reduced conjugate may be an important determinant of toxicity and future experiments could be designed to identify the specific transporter(s) involved in the extrusion of not only the glutathione conjugates, but base propenol as well. Importantly, the elucidation of the cellular metabolism will permit further studies on whole organ or animals. c) Glutathione conjugates are rapidly extruded from the cells. In situ, these conjugates are converted into mercapturic acids and then excreted in the urine. If AR is an important component, the predominant form of the metabolite in the urine will be the mercapturic acid analog of adenine propenol. Quantification of this metabolite will provide a measure of on-going DNA damage in the organism. These measurements may also be useful in non-invasively quantifying DNA damage in humans. Such measurements may be particularly relevant in identifying individual sensitivity to environmental or drug-induced DNA damage. d) Investigating the in vivo role of AR in detoxifying the base propenals, regulating the formation of M₁G adduct and modulating BLM toxicity. e) When experiments show that AR protects against base propenal and BLM toxicity, the specific events in the apoptotic or necrotic pathway that are affected by AR will be elucidated. This should result in a deeper understanding of the mechanisms by which base propenals and BLM cause cell death. f) Test whether the expression of AR affects the BLM-sensitivity of specific tumors, whether AR overexpressing tumors are more refractory to BLM. g) The use of compounds identified from analogs to glutathione conjugates in detecting of AR or screening of AR activities in samples obtained from patients and determining the disease conditions. h) Finalfy, the lead compounds discovered in this study will require complete and thorough clinical trials for their use against diseases such as cancer.

SEQ ID NO:1
Human Aldose Reductase
Primary Accession Number in SWISS-PROT: P15121

```
      10 20 30 40 50 60
      | | | | | |
ASRLLLNNGA KMPILGLGTW KSPPGQVTEA VKVAIDVGYR
HIDCAHVYQN ENEVGVAIQE
      70 80 90 100 110 120
      | | | | | |
KLREQVVKRE ELFIVSKLWC TYHEKGLVKG ACQKTLSDLK
LDYLDLYLIH WPTGFKPGKE
      130 140 150 160 170 180
      | | | | | |
FFPLDESGNV VPSDTNILDT WAAMEELVDE GLVKAIGISN
FNHLQVEMIL NKPGLKYKPA
      190 200 210 220 230 240
      | | | | | |
VNQIECHPYL TQEKLIQYCQ SKGIVVTAYS PLGSPDRPWA
KPEDPSLLED PRIKAIAAKH
      250 260 270 280 290 300
      | | | | | |
NKTTAQVLIR FPMQRNLVVI PKSVTPERIA ENFKVFDFEL
SSQDMTTLLS YNRNWRVCAL
      310
      |
LSCTSHKDYP FHEEF
```

References Cited in this Application
1. Gabbay, K. H. The sorbitol pathway and the complications of diabetes. N Engl J Med 288:831-6, (1973).
2. Gabbay, K. H. Hyperglycemia, polyol metabolism, and complications of diabetes mellitus. Annu Rev Med 26:521-36, (1975).
3. Wilson, D. K., Bohren, K. M., Gabbay, K. H. and Quiocho, F. A.: An unlikely sugar substrate site in the 1.65 A structure of the human aldose reductase holoenzyme implicated in diabetic complications. Science 257:81-84, (1992).
4. Rondeau, J. M., Tete-Favier, F., Podjarny, A., Reymann, J. M., Barth, P., Biellmann, J. F. and Moras, D.: Novel NADPH-binding domain revealed by the crystal structure of aldose reductase. Nature 355:469-472, (1992).
5. Borhani, D. W., Harter, T. M. and Petrash, J. M.: The crystal structure of the aldose reductase. NADPH binary complex. J Biol Chem 267:24841-7, (1992).
6. Wilson, D. K., Tarle, I., Petrash, J. M. and Quiocho, F. A.: Refined 1.8 A structure of human aldose reductase complexed with the potent inhibitor zopolrestat. Proc Natl Acad Sci USA 90:9847-51, (1993).
7. Harrison, D. H., Bohren, K. M., Ringe, D., Petsko, G. A. and Gabbay, K. H.: An anion binding site in human aldose reductase: mechanistic implications for the binding of citrate, cacodylate, and glucose 6-phosphate. Biochemistry 33:2011-20, (1994).
8. Bohren, K. M., Grimshaw, C. E., Lai, C. J., Harrison, D. H., Ringe, D., Petsko, G. A. and Gabbay, K. H.: Tyrosine-48 is the proton donor and histidine-110 directs substrate stereochemical selectivity in the reduction reaction of human aldose reductase: enzyme kinetics and crystal structure of the Y48H mutant enzyme. Biochemistry 33:2021-32, (1994).
9. Urzhumtsev, A., Tete-Favier, F., Mitschler, A., Barbanton, J., Barth, P., Urzhumtseva, L., Biellmann, J. F., Podjarny, A. and Moras, D.: A ‘specificity’ pocket inferred from the crystal structures of the complexes of aldose reductase with the pharmaceutically important inhibitors tolrestat and sorbinil. Structure 5:601-12, (1997).
10. Harrison, D. H., Bohren, K. M., Petsko, G. A., Ringe, D. and Gabbay, K. H.: The alrestatin double-decker: binding of two inhibitor molecules to human aldose reductase reveals a new specificity determinant. Biochemistry 36:16134-40, (1997).
11. Oka, M., Matsumoto, Y., Sugiyama, S., Tsuruta, N. and Matsushima, M.: A Potent Aldose Reductase Inhibitor, (2S, 4S)-6-Fluoro-2′5′-Dioxospiro[Chroman-4,4′-Imidazolidine]-2-Carboxamide(Fidarestat): Its Absolute Configuration and Interactions with the Aldose Reductase by X-Ray Crystallography J. Med. Chem. 43:2479-83, (2000).
12. Calderone, V., Chevrier, B., Van Zandt, M., Lamour, V., Howard, E., Poterzman, A., Barth, P., Mitschler, A., Lu, J., Dvornik, D. M., Klebe, G., Kraemer, O., Moorman, A. R., Moras, D. and Podjarny, A.: The Structure of Human Aldose Reductase Bound to the Inhibitor Idd384 Acta Crystallogr., Sect. D 56:536-40, (2000).
13. Srivastava, S., Chandra, A., Bhatnagar, A., Srivastava, S. K. and Ansari, N. H. Lipid peroxidation product, 4-hydroxynonenal and its conjugate with GSH are excellent substrates of bovine lens aldose reductase. Biochem. Biophys. Res. Commun. 217:741-746, (1995).
14. Srivastava S., Chandra, A., Ansari, N. H., Srivastava, S. K. and Bhatnagar, A. Identification of cardiacoxidoreductase(s) involved in the metabolism of the lipid peroxidation derived aldehyde, 4-hydroxynonenal. Biochem. J. 329:469-475, (1998).
15. Srivastava S., Chandra, A., Wang, L., Seifert, W. E. Jr., Dague, B. B., Ansari, N. H., Srivasrava S. K. and Bhatnagar, A. Metabolism of lipid peroxidation product, 4-hydroxy trans-2-nonenal in isolated rat heart. J. Biol. Chem. 273:10893-10890, (1998).
16. Srivastava, S., Harter, T. M., Chandra, A., Bhatnagar, A, Srivastava, S. K. and Petrash, J. M. Kinetic studies of a FR-1, a growth factor-inducible aldo-keto reductase. Biochemistry 37:12909-12917, (1998).
17. He, Q., Khanna, P., Srivastava, S., van Kuijk, F. J. G. M. and Ansari, N. H. Reduction of 4-hydroxynonenal and 4-hydroxy hexenal by retinal aldose reductase. Biochem. Biophys. Res Commun. 247:719-722, (1998).
18. Srivastava, S., Watowich. S., Petrash, J. M., Srivastava, S. K. and Bhatnagar, A. Structural and kinetic properties of aldehyde reduction by aldose reductase. Biochemistry 38:42-54, (1999).
19. Ruef, J., Liu, S. -Q., Bode, C., Tocchi, M., Srivastava, S., Runge, M. S. and Bhatnagar, A. Involvement of aldose reductase in vascular smooth muscle growth and lesion formation following arterial injury. Arterio. Thromb. Vasc. Biol. 20:1745-1752, (2000).
20. Dixit, B. L., Balendrian, G. K., Watowich, S. J., Srivastava, S., Ramana, K. V., Petrash, J. M., Bhatnagar, A. and Srivastava, S. K. Kinetic and structural characterization of the glutathione binding site of aldose reductase. J. Biol. Chem. 275:21587-21595, (2000).
21. Nath, R. G. and Chung, F. L. Detection of exocyclic 1,N2-propanodeoxyguanosine adducts as common DNA lesions in rodents and humans. Proc Natl Acad Sci U S A. 91:7491-5, (1994).
22. Trush, M. A. and Kensler, T. W. An overview of the relationship between oxidative stress and chemical carcinogenesis. Free Radic. Biol. Med. 10:201-209, (1991).
23. Poulsen, H. E., Prieme, H. and Loft, S. Role of oxidative DNA damage in cancer initiation and promotion Eur. J. Cancer Prev. 7:9-16, (1998).
24. Esterbauer, H., Schaur, R. J. and Zollner, H. Chemistry and biochemistry of 4-hydroxynonenal, malonaldehyde and related aldehydes. Free Radic. Bio. Med. 11:81-128, (1991).
25. Rao, M. S. and Olson, A. J. Modelling of factor Xa-inhibitor complexes: a computational flexible docking approach. Prot Struct Funct Gen 34:173-83, (1999).
26. Zeindl-Eberhart, E., Jungblut, P. R., Otto, A., Kerler, R. and Rabes, H. M. Further characterization of a rat hepatoma-derived aldose-reductase-like protein—organ distribution and modulation in vitro. Eur J Biochem. 247:792-800, (1997).
27. Zeindl-Eberhart, E., Jungblut, P. R., Otto, A. and Rabes, H. M. Identification of tumor-associated protein variants during rat hepatocarcinogenesis. Aldose reductase. J Biol Chem. 269:14589-14594, (1994).
28. Cao, D., Fan, S. T. and Chung, S. S. Identification and characterization of a novel human aldose reductase-like gene. J Biol Chem. 273:11429-11435, (1998).
29. Ahmed, N. K., Felsted, R. L. and Bachur, N. R. Daunorubicin reduction mediated by aldehyde and ketone reductases. Xenobiotica. 11:131-136, (1981).
30. Rittner, H. L., Hafner, V., Klimiuk, P. A., Szweda, L. I., Goronzy, J. J. and Weyand, C. M. Aldose reductase functions as a detoxification system for lipid peroxidation products in vasculitis. J Clin Invest. 103:1007-13, (1999).
31. Grollman, A. P. Base propenals and toxicity of bleomycin. Dev Oncol. 53:79-90, (1988).
32. Grollman, A. P., Takeshita, M., Pillai, K. M. R. and Johnson, F. Origin and cytotoxic properties of base propenals derived from DNA. Cancer Res 45:1127-1131, (1985).
33. Kalman, T. I., Marinelli, E. R., Xu, B., Venugopala, R., Johnson, F. and Grollman, A. P. Inhibition of cellular thymidylate synthesis by cytotoxic propenal derivatives of pyrimidine bases and deoxynucleosides. Biochem Pharmacol 42:431-437, (1991).
34. Esterbauer, H., Schaur, R. J. and Zollner, H. Chemistry and biochemistry of 4-hydroxynonenal, malonaldehyde and related aldehydes. Free Radic. Bio. Med. 11:81-128, (1991).
35. Balendiran, G. K., Schnutgen, F., Scapin, G., Borchers, T., Xhong, N., Godbout, R., Spener, F. and Sacchettini, J. C. Crystal Structure and thermodynamic analysis of human brain fatty acid binding protein. J. Biol. Chem. 275:27045-27054, (2000).
36. Balendiran, G. K., Molina, J. A., Xu, Y., Torres-Martinez, J., Stevenes, R., Focia, P. J., Eakin, A. E., Sacchettini, J. C and Craig III, S. P. Ternary complex structure of human HGPRTase, PRPP, Mg²⁺, and the inhibitor HPP reveals the involvement of the flexible loop in substrate binding. Protein Science, 8:1023-1031, (1999).
37. Balendiran, G. K., Solieim, J. C., Young, A. C. M., Hansen, T. H., Nathanson, S. G. and Sacchettini, J. C. The three-dimensional structure of an H-2L^{d}-peptide complex explains the unique interaction of L^{d} with beta-2 microglobulin and peptide. Proc Natl Acad Sci USA, 94:6880-6885, (1997).
38. Hornell, T., Wormstall, E. M., Russell, J., Balendiran, G. K., Connolly, J. M., Cook, J. R. and Hansen, T. H. T-cell recognition of class I MHC H-2Ld and the residues involved in the interaction. J Immuno., 163:3217-3225, (1999).
39. Yu, Y. Y., Turnquist, H. R., Myers, N. B., Balendiran, G. K., Hansen, T. H. and Solheim, J. C. An extensive region of an MHC class I alpha2 domain loop influences interaction with the assembly complex. J. Immuno., 163:4427-4433, (1999).
40. Yu, Y. Y., Myers, N. B., Hilbert, C. M. Harris, M. R., Balendiran, G. K. and Hansen, T. H. Definition and transfer of an epitope specific for peptide empty forms of MHC class I. International Immunology, 11:1897-1905, (1999).
41. Ramana, K. V., Dixit, B. L., Srivastava, S., Balendiran, G. K., Srivastava, S. K. and Bhatnagar, A. Selective recognition of glutathiolated aldehydes by aldose reductase. Biochemistry 39:12172-12180, (2000).
42. Ramana, K. V., Dixit, B.L., Srivastava, S., Bhatnagar, A., Balendiran, G. K., Watowich, S. J., Petrash, J. M. and Srivastava, S. K. Characterization of the glutathione binding site of aldose reductase. Chem Biol Interact. 130-132:537-548, (2001).
43. Murugesan, N., Xu, C., Ehrenfeld, G. M., Sugiyama, H., Kilkuskie, R. E., Rodriguez, L. O., Chang, L. H. and Hecht, S. M. Analysis of products formed during bleomycin-mediated DNA degradation. Biochemistry 24:5735-44, (1985).
44. Vander Jagt, D. L., Kolb, N. S., Vander Jagt, T. J., Chino, J., Martinez, F. J., Hunsaker, L. A. and Royer, R. E. Substrate specificity of human aldose reductase: identification of 4-hydroxynonenal as an endogenous substrate. Biochim Biophys Acta 1249:117-26, (1995).
45. Srivastava, S., Liu, S. Q., Conklin, D. J., Zacarias, A., Srivastava, S. K. and Bhatnagar, A. Involvement of aldose reductase in the metabolism of atherogenic aldehydes. In: Enzymology and Molecular Biology of Carbonyl Metabolism, H. Weiner Edition, Amsterdam: 563-571, (2001).
46. Haddad, J., Decker, M. L., Hsieh, L. C., Lesch, M., Samarel, A. M. and Decker, R. S. Attachment and maintenance of adult rabbit cardiac myocytes in primary cell culture. Am. J. Physiol.; 255:C19-C27, (1988).
47. Horvath, J. J., Witmer, C. M. and Witz, G. Nephrotoxicity of the 1:1 acrolein-glutathione adduct in the rat. Toxicol Appl Pharmacol 117:200-207, (1992).
48. Wang, H. G., Rapp, U. R. and Reed, J. C. BC1-2 targets the protein kinase Raf-1 to mitochondria. Cell 87:629-638, (1996).
49. Richter, C., Park, J. W. and Ames, B. N. Normal oxidative damage to mitochondrial and nuclear DNA is extensive. Proc. Natl. Acad. Sci. U.S.A. 85:6465-6467, (1988).
50. Beckman, K. B and Ames, B. N. Oxidative decay of DNA. J. Biol. Chem. 272:19633-19636, (1997).
51. Povirk, L. F. DNA damage and mutagenesis by radiomimetic DNA-cleaving agents: bleomycin, neocarzinostatin and other enediynes. Mutat. Res., 355:71-89, (1996).
52. Dahm-Daphi, J., Sass, C. and Alberti, W. Comparison of biological effects of DNA damage induced by ionizing radiation and hydrogen peroxide in CHO cells. Int. J Radiat. Biol. 76:67-75, (2000).
53. Chaudhry, M. A. and Weinfeld, M. Reactivity of human apurinic/apyrimidinic endonuclease and Escherichia coli exonuclease III with bistranded abasic sites in DNA. J. Biol. Chem., 272:15650-15655, (1997).
54. Ames, B. N. Cancer prevention: novel nutrient and pharmaceutical developments. Ann. N. Y. Acad. Sci., 889:87-106, (1999).
55. Pfeiffer, P. The mutagenic potential of DNA double-strand break repair. Toxicol. Lett., 96/97:119-129, (1998).
56. Pfeifer, M. A., Schumer, M. P. and Gelber, D. A. Aldose reductase inhibitors: the end of an era or the need for different trial designs? Diabetes 46 Suppl 2:S82-9, (1997).
57. Calabresi P., Schein, P. S. and Rosenberg, S. A. Medical Oncology, Macmillan Publishing Co., New York, (1985).
58. Dedon, P. C. and Goldberg, I. H. Free-radical mechanisms involved in the formation of sequence-dependent bistranded DNA lesions by the antitumor antibiotics bleomycin, neocarzinostatin, and calicheamicin. Chem. Res. Toxicol. 5:311-332, (1992).
59. Barciszewski, J., Siboska, G. E., Pedersen, B. O., Clark, B. F. and Rattan, S. 1. Furfural, a precursor of the cytokinin hormone kinetin, and base propenals are formed by hydroxyl radical damage of DNA. Biochem. Biophys Res Comm; 238:317-319, (1997).
60. Yermilov, V., Yoshie, Y., Rubio, J. and Ohshima, H. Effects of carbon dioxide/bicarbonate on induction of DNA single-strand breaks and formation of 8-nitroguanine, 8-oxoguanine and base-propenal mediated by peroxynitrite. FEBS Lett.; 399:67-70, (1996).
61. Casadevall, M., da Cruz, F. P. and Kortenkamp, A. Chromium(VI)-mediated DNA damage: oxidative pathways resulting in the formation of DNA breaks and abasic sites. Chem. -Biol. Interact. 123:117-132, (1999).
62. Rashid, R., Langfinger, D., Wagner, R., Schuchmann, H. P. and von Sonntag, C. Bleomycin versus OH-radical-induced malonaldehydic-product formation in DNA. Int. J. Radiat. Biol. 75:101-109, (1999).
63. Arrick, B. A. and Nathan, C. F. Glutathione metabolism as a determinant of therapeutic efficacy: a review. Cancer Res, 33:4224, (1984).
64. Mantle, T. J., McCusker, F. M., Phillips, M., Boyce, S. Glutathione S-transferases Biochem Soc Trans Isoenzymes 18:175-7, (1990).
65. Mannervik, B., Awasthi, Y. C., Board, P. G., Hayes, J. D., Di Ilio, C., Ketterer, B., Listowsky, I., Morgenstern, R., Muramatsu, M., Pearson, W. R., et al., Nomenclature for human glutathione transferases Biochem J. 282:305-6, (1992).
66. Mulder, T. P., Manni, J. J., Roelofs, H. M., Peters, W. H., Wiersma, A. Glutathione S-transferases and glutathione in human head and neck cancer. Carcinogenesis 16: 619-24, (1995).
67. Ribrag, V., Massaad, L., Janot, F., Morizet, J., Gouyette, A. and Chabot, G. G. Main drug-metabolizing enzyme systems in human non-Hodgkin's lymphomas sensitive or resistant to chemotherapy. Leuk Lymphoma. 18:303-10, (1995).
68. Takahashi, M., Fujii, J., Miyoshi, E., Hoshi, A. and Taniguchi, N. Elevation of aldose reductase gene expression in rat primary hepatoma and hepatoma cell lines: implication in detoxification of cytotoxic aldehydes. Int. J Cancer. 62:749-754 (1995).
69. Hedberg, J.J., Grafstrom, R. C., Vondracek, M., Sarang, Z., Warngard, L. and Hoog, J. O. Micro-array chip analysis of carbonyl-metabolising enzymes in normal, immortalised and malignant human oral keratinocytes. Cell Mol Life Sci. 58: 1719-26, (2001).
70. Scuric, Z., Stain, S. C., Anderson, W. F. and Hwang, J. J. New member of aldose reductase family proteins overexpressed in human hepatocellular carcinoma. Hepatology 27:43-50, (1998).
71. Ramana, K. V., Chandra, D., Srivastava, S., Bhatnagar, A., Aggarwal, B.B. and Srivastava, S. K. Aldose reductase mediates mitogenic signaling in vascular smooth muscle cells. J. Biol. Chem. 277:32063-70, (2002).
72. Meister, A. Glutathione deficiency produced by inhibition of its synthesis, and its reversal; applications in research and therapy. Pharmacol Ther. 51:155-94, (1991).
73. Mitchell, J. B. and Russo, A. The role of glutathione in radiation and drug induced cytotoxicity. Br J Cancer Suppl. 8:96-104, (1987).
74. Estrela, J. M., Obrador, E., Navarro, J., Lasso De la Vega, M. C. and Pellicer, J. A Elimination of Ehrlich tumours by ATP-induced growth inhibition, glutathione depletion and X-rays. Nat Med. 1:84-8, (1995).
75. Navarro, J., Obrador, E., Carretero, J., Petschen, I., Avino, J., Perez, P. and Estrela, J. M. Changes in glutathione status and the antioxidant in blood and in cancer cells associate with tumour growth in vivo. Free Radic Biol Med. 26:410-8, (1999).
76. Bradford, M. M. A rapid and sensitive method for the quantitation of microgram quantities of protein utilizing the principle of protein-dye binding. Anal Biochem. 72:248-254, (1976).
77. Ward, L. D. Measurement of ligand binding to proteins by fluorescence spectroscopy. Methods Enzymol. 117:400-414, (1985).
78. Cleland, W. W. Statistical analysis of enzyme kinetic data. Methods Enzymol. 63:103-138, (1979).
79. Hohman, T. C., E1-Kabbani, O., Malamas, M. S., Lai, K., Putilina, T., McGowan, M. H., Wane, Y. Q. and Carper, D. A. Probing the inhibitor-binding site of aldose reductase with site-directed mutagenesis. Eur J Biochem. 256:310-6, (1998).
80. Tsai, W. Y., Chang, W. H., Chen, C. H. and Lu, F. J. Enchancing effect of patented whey protein isolate (Immunocal) on cytotoxicity of an anticancer drug. Nutr Cancer. 38:200-8, (2000).
81. Meister, A. Selective modification of glutathione metabolism. Selective modification of glutathione metabolism. Science. 220:472-7, (1983).
82. Mitchell, J. B., Cook, J. A., DeGraff, W., Glatstein, E. and Russo, A. Glutathione modulation in cancer treatment: will it work? Int J Radiat Oncol Biol Phys. 16:1289-95, (1989).
83. Shaw, J. P. and Chou, I. -N. Elevation of intracellular glutathione content associated with mitogenic stimulation of quiescent fibroblasts. J. Cell. Physiol. 129,193-198, (1986).
84. Suthanthiran, M., Anderson, M. E., Sharma, V. K. and Meister, A. Glutathione regulates activation-dependent DNA synthesis in highly purified normal human T lymphocytes stimulated via the CD2 and CD3 antigens. Proc Natl Acad Sci U S A. 87:3343-7, (1990).
85. Terradez, P., Asensi, M., Lasso de la Vega, M. C., Puertes, I., Viña, J. and Estrela, J. M. Depletion of tumour glutathione in vivo by buthionine sulphoximine: modulation by the rate of cellular proliferation and inhibition of cancer growth. J. Biochem. 292:477-483, (1993).
86. Estrela, J. M., Hernandez, R., Terradez, P., Asensi, M., Puertes, I. R. and Viña, J. Regulation of glutathione metabolism in Ehrlich ascites tumour cells. J. Biochem. 286:257-262, (1992).
87. Colell, A., Garcia-Ruiz, C., Miranda, M., Ardite, E., Mari, M., Morales, A., Corrales, F., Kaplowitz, N. and Fernandez-Checa, J. C. Selective glutathione depletion of mitochondria by ethanol sensitizes hepatocytes to tumor necrosis factor. Gastroenterology 115:1541-51, (1998).
88. Smardo, F. L. Jr, Burg, M. B. and Garcia-Perez, A. Kidney aldose reductase gene transcription is osmotically regulated. Am J Physiol. 262:C776-82, (1992).
89. Garcia-Perez, A., Martin, B., Murphy, H. R., Uchida, S., Murer, H., Cowley, B. D. Jr, Handler, J. S. and Burg, M. B. Molecular cloning of cDNA coding for kidney aldose reductase. Regulation of specific mRNA accumulation by NaCl-mediated osmotic stress. J Biol Chem. 264:16815-21, (1989).
90. Moriyama, T., Garcia-Perez, A. and Burg, M. B. Osmotic regulation of aldose reductase protein synthesis in renal medullary cells. J Biol Chem. 264:16810-4, (1989).
91. Uchida, S., Garcia-Perez, A., Murphy, H. and Burg, M. Signal for induction of aldose reductase in renal medullary cells by high external NaCl. Am J Physiol 256:C614-20, (1989).
92. Chou, T. C. and Talalay, P. Generalized equations for the analysis of inhibitions of Michaelis-Menten and higher-order kinetic systems with two or more mutually exclusive and nonexclusive inhibitors. Eur. J. Biochem. 115:207-216, (1981).
93. Lamour, V., Barth, P., Rogniaux, H., Poterszman, A., Howard, E., Mitschler, A., Van Dorsselaer, A., Podjarny, A. and Moras, D. Production of crystals of human aldose reductase with very high resolution diffraction. Acta Crystallogr D55:721-3, (1999).
94. Johnson, F., Pillai, K. M. R., Grollman, A. P., Tseng, L. and Takeshita, M. Synthesis and biological activity of a new class of cytotoxic agents: N-(3-oxopro-1-enyl)-substituted pyrimidines and purines. J. Med. Chem. 27:954-958, (1984).
95. Brownlee, M. Biochemistry and molecular cell biology of diabetic complications. Nature 414:813-20, (2001).
96. Jez, J. M., Schlegel, B. P and Penning, T. M. Characterization of the substrate binding site in rat liver 3alpha-hydroxysteroid/dihydrodiol dehydrogenase. The roles of tryptophans in ligand binding and protein fluorescence. J Biol Chem 271:30190-8, (1996).
97. Penning, T. M. Molecular determinants of steroid recognition and catalysis in aldo-keto reductases. Lessons from 3 alpha-hydroxysteroid dehydrogenase. J Steroid Biochem Mol Biol. 69:211-25, (1999).
98. Petrash, J. M., Murthy, B. S., Young, M., Morris, K., Rikimaru, L., Griest, T. A. and Harter, T. Functional genomic studies of aldo-keto reductases. Chem Biol Interact. 130-132:673-683, (2001).
99. Srivastava, S., Chandra, A., Wang, L. F., Seifert, W. E. Jr., DaGue, B. B., Ansari, N. H., Srivastava, S. K. and Bhatnagar, A. Metabolism of the lipid peroxidation product, 4-hydroxy trans-2-nonenal, in isolated perfused rat heart. J. Biol Chem. 273:10893-10900, (1998).
100. Lindahl, T. Instability and decay of the primary structure of DNA. Nature 362: 709-715, (1993).
101. Wang, J., Chen, W., Tsai, S., Sung, P. and Huang, R. An in vitro model for evaluation of vaporous toxicity of trichloroethylene and tetrachloroethylene to CHO-K1 cells. Chem Biol Interact. 137:139-54, (2001).
102. SYBYL, Tripos Inc.; St. Louis, Mo., USA, (1998).
103. Kao, Y. L., Donaghue, K., Chan, A., Knight, J. and Silink, M. A novel polymorphism in the aldose reductase gene promoter region is strongly associated with diabetic retinopathy in adolescents with type 1 diabetes. Diabetes 48:1338-1340, (1999).
104. Cramer III, R. D., Patterson, D. E. and Bunce, J. D. Comparative Molecular Field Analysis (CoMFA). Effect of shapes on binding of steroids to carrier proteins. J. Amer. Chem. Soc., 110:5959-5967, (1988).
105. Lemmen, C., Lengauer, T. and Klebe, G. FLEXS: a method for fast flexible ligand superposition. J. Med. Chem. 41:4502-20, (1998).
106. Goodsell, D. S., Morris, G. M. and Olson, A. J. Automated docking of flexible ligands: applications of AutoDock J. Mol. Recognit. 9:1-5, (1996).
107. Bailey, H. H., Gipp, J. J., Ripple, M., Wilding, G. and Mulcahy, R. T. Increase in gamma-glutamylcysteine synthetase activity and steady-state messenger RNA levels in melphalan-resistant DU-145 human prostate carcinoma cells expressing elevated glutathione levels. Cancer Res 52:5115-8, (1992).
108. Godwin, A. K., Meister, A., O'Dwyer, P. J., Huang, C. S., Hamilton, T. C. and Anderson, M. E. High resistance to cisplatin in human ovarian cancer cell lines is associated with marked increase of glutathione synthesis. Proc Natl Acad Sci U S A. 89:3070-4, (1992).
109. Rudra, P. K. and Krokan, H. E. Acrolein cytotoxicity and glutathione depletion in n-3 fatty acid sensitive- and resistant human tumor cells. Anticancer Res. 19:461-9, (1999).
110. Jancarik, J. and Kim, S. -H. Sparse matrix sampling: A screening method for crystallization of proteins. J. Appl. Cryst. 24:409-411, (1991).
111. Navaza, J. AMoRe: an automated package for molecular replacement. Acta Crystallogr. A50:157-160, (1994).
112. Brünger, A. T. X-PLOR Manual, Version 3.1, Yale University, New Haven, Conn., (1992).
113. Brünger, A. T., Adams, P. D., Clore, G. M., Gros, P., Grosse-Kunstleve, R. W., Jiang, J. -S., Kuszewski, J., Nilges, N., Pannu, N. S., Read, R. J., Rice, L. M., Simonson, T. and Warren, G. L. Crystallography & NMR system (CNS): A new software system for macromolecular structure determination, Acta Cryst. D54:905-921, (1998).
114. Cheng, X., Balendiran, K., Schildkrault, I. and Anderson, J. E. Structure of PvuII endonuclease with cognate DNA. EMBO J 13:3927-3935, (1994).
115. Stout, G. H. and Jensen, L. H. X-ray structure determination. New York: Wiley Interscience, (1989).
116. Blundell, T. L. and Johnson, L. N. Protein Crystallography, Academic Press London 375-377, (1976).
117. Hendrickson, W. A. Determination of macromolecular structures from anomalous diffraction of synchrotron radiation. Science 254:51-58, (1991).
118. Balendiran, K., Bonventre, J., Knott, R., Jack, W., Benner, J., Schildkraut, I., and Anderson, J. E. Expression, purification, and crystallization of restriction endonuclease PvuII with DNA containing its recognition site. Proteins: Structure, Function, and Genetics 19:77-79, (1994).
119. Nishikawa T, Edelstein D, Du X L, Yamagishi S, Matsumura T, Kaneda Y, Yorek M A, Beebe D, Oates P J, Hammes H P, Giardino I, Brownlee M. Normalizing mitochondrial superoxide production blocks three pathways of hyperglycaemic damage. Nature 404:787-90, (2000).
120. Goodford, P. J. A computational procedure for determining energetically favorable binding sites on biologically important macromolecules. J. Med Chem 28:849-57, (1985).
121. Schapira, M., Raaka, B. M., Samuels, H. H. and Abagyan, R. Rational discovery of novel nuclear hormone receptor antagonists. Proc Natl Acad Sci U S A. 97:1008-13, (2000).
122. Necessary components with suppliers or references for preparation: glutathione, glutamic acid, cysteine, glycine, N-methylglycine, ethanolamine, aspartic acid, and acrolein, Aldrich; 2-hydroxypentanedioic acid and N-methylglutamic acid, Sigma; (S)-4-amino-5-hydroxypentanoic acid. Barlos, K., Mamos, P., Papaioannou, D. and Patrianakou, S. Syntheses of (S)-2- and 4-amino-5-hydroxypentanoic acids. J. Chem. Soc., Chem. Commun. 1583-1584, (1987); N,N-dimethylglutamic acid. Ikutani, Y. N-Oxides of N,N-dialkylamino acids. III. Syntheses of N,N-dimethyl acidic amino acids and related compounds. Bull. Chem. Soc. Jpn. 43:3602-3603, (1970); N-methylcysteine. Menger, F. M. and Caran, K. L. Anatomy of a Gel. Amino Acid Derivatives That Rigidify Water at Submillimolar Concentrations. J. Am. Chem. Soc. 122:11679-11691, (2000).
123. Necessary components with suppliers or references for preparation: cinnamaldehyde, Aldrich; 3-(2-furyl)propenal, ACROS; 3-(4-pyridyl)propenal, TCI America; □□ -phenylacrolein, Laitalainen, T., Kuronen, P. and Hesso, A. A one-step preparation and hetero-Diels Alder dimerization of 2-phenylpropenal. Org. Prep. Proc. Int. 25:597-599, (1993); 2-(2-furyl)-3-chloropropene, Bargar, T. M., Broersma, R. and McCarthy, J. R. Preparation of beta-methylenefuranethanamines, inhibitors of dopamine beta-hydroxylase, and their use as antihypertensives. CA 106:50015-19, (1986); 2-(4-pyridyl)-2-propen-1-ol, Koei Chemical Co., LTD.
124. Li, Y., Carter, D. E. and Mash, E. A. Synthesis and structure of the glutathione conjugate of chloroacetaldehyde. Synthetic Communications 32:1579-1583, (2002).
125. So, S. S. and Karplus, M. Evaluation of designed ligands by a multiple screening method: application to glycogen phosphorylase inhibitors constructed with a variety of approaches. J. Comput Aided Mol 15:613-47, (2001).
126. Sippl W. Receptor-based 3D QSAR analysis of estrogen receptor ligands—merging the accuracy of receptor-based alignments with the computational efficiency of ligand-based methods. J. Comput Aided Mol Des. 14:559-72, (2000).
127. Goldblatt S A, Nadel E M. Cancer cells in the circulating blood: a critical review. Acta Cytol 1965; 305: 6-20.
128. Glaves D. Correlation between circulation cancer cells and incidence of metastases. Br J Cancer 1983; 48: 665-673.
129. Fidler I J. The biology of human cancer metastasis. 7th Jan Waldenstron lecture. Acta Oncol 1991; 30: 668-675.
130. Raper S R. Answering questions on a microscopic scale: the detection of circulation cancer cells. Surgery 1999; 126: 827-828.
131. Lee C S, Hwang L Y, Beasely R P, Hsu H C, Lee H S, Lin T Y. Prognostic significance of histologic findings in resected small hepatocellular carcinoma. Acta Chir Scand 1988; 154: 199-203.
132. Lai E C, Ng I O, Ng M M, Lok A S, Tam P C, Fan S T, Choi T K, Wong J. Long term results of resection for large hepatocellular carcinoma: A multivariate analysis of clinicopathological features. Hepatology 1990; 11: 815-818.
133. Jeng K S, Chen B F, Lin H J. En bloc resection for extensive hepatocellular carcinoma:

Is it advisable? World J Surg 1994; 18: 834-839.
134. Alter M J, Kruszon-Moran D, Nainan O V, et al. The prevalence of hepatitis C virus infection in the United States, 1988 through 1994. N Engl J Med 1999;341:556-62.
135. Tang B, Kruger W D, Chen G, Shen F, Lin W Y, Mboup S, London W T, Evans A A. J Med Virol. 2004 Jan;72(1):35-40.
136. Takahashi M, Hoshi A, Fujii J, Miyoshi E, Kasahara T, Suzuki K, Aozasa K, Taniguchi N. Jpn J Cancer Res. 1996 Apr;87(4):337-41.
137. Zhou XD. Hepatobiliary Pancreat Dis Int. 2002 Feb;1(1):35-41.
138. Dewys W D, Begg C, Lavin P T, Band P R, Bennett J M, Bertino J R, Cohen M H, Douglass H O Jr, Engstrom P F, Ezdinli E Z, Horton J, Johnson G J, Moertel C G, Oken M M, Perlia C, Rosenbaum C, Silverstein M N, Skeel R T, Sponzo R W, Tormey D C. Prognostic effect of weight loss prior to chemotherapy in cancer patients. Eastern Cooperative Oncology Group. Am J Med. 1980 October;69(4):491-7.
139. Kern K A, Norton J A. Cancer cachexia. JPEN J Parenter Enteral Nutr. 1988 May-June;12(3):286-98.
140. Curtis E B, Krech R, Walsh T D. Common symptoms in patients with advanced cancer. J Palliat Care. 1991 Summer;7(2):25-9.
141. Lazo P A. Tumour-host metabolic interaction and cachexia. FEBS Lett. 1985 August 5;187(2):189-92.
142. Legaspi A, Jeevanandam M, Starnes H F Jr, Brennan M F. Whole body lipid and energy metabolism in the cancer patient. Metabolism. 1987 October;36(10):958-63.
143. Mulligan H D, Beck S A, Tisdale M J. Lipid metabolism in cancer cachexia. Br J Cancer. 1992 July;66(1):57-61.
144. Kawamura I, Yamamoto N, Sakai F, Yamazaki H, Goto T. Anticancer Res. 1999 September-October; 19(5B):4099-103
145. Kawamura I, Yamamoto N, Sakai F, Yamazaki H, Naoe Y, Inami M, Manda T, Shimomura K. Activation of lipoprotein lipase and inhibition of B 16 melanoma-induced cachexia in mice by ponalrestat, an aldose reductase inhibitor. Anticancer Res. 1999 January-February;19(1A):341-8.
146. Kawamura I, Lacey E, Inami M, Nishigaki F, Naoe Y, Tsujimoto S, Manda T, Goto T. Ponalrestat, an aldose reductase inhibitor, inhibits cachexia syndrome in nude mice bearing human melanomas G361 and SEKI. Anticancer Res. 1999 September-October; 19(5B):4091-7.
147. Kawamura I, Lacey E, Yamamoto N, Sakai F, Takeshita S, Inami M, Nishigaki F, Naoe Y, Tsujimoto S, Manda T, Shimomura K, Goto T. Anticancer Res. 1999 September-October; 19(5B):4105-11.
148. Nishimura C, Matsuura Y, Kokai Y, Akera T, Carper D, Morjana N, Lyons C, Flynn T G. Cloning and expression of human aldose reductase. J. Biol Chem. 1990 June 15;265(17):9788-92.
149. Bando T, Fujimura M, Kasahara K, Shibata K, Shirasaki H, Heki U, Iwasa K, Ueda A, Tomikawa S, Matsuda T. (1997). Exposure to sorbitol induces resistance to cisplatin in human non-small-cell lung cancer cell lines. Anticancer Res, 17: 3345-3348
150. Ferretti A, D'Ascenzo S, Knijn A, Iorio E, Dolo V, Pavan A, Podo F. Detection of polyol accumulation in a new ovarian carcinoma cell line, CABA I: a(1)H NMR study. Br J Cancer. 2002 April 8;86(7):1180-7.
151. Zeindl-Eberhart E, Haraida S, Liebmann S, Jungblut P R, Lamer S, Mayer D, Jager G, Chung S, Rabes H M. Detection and identification of tumor-associated protein variants in human hepatocellular carcinomas. Hepatology. 2004 February ;39(2):540-9.
152. Mylari et al., J. Med. Chem (1991) 34(3): 1011-8.
153. Lee, Inhibitory Activity of Cinnamomum cassia Bark-Derived Component against Rat Lens aldose Reductase, J. Pharm Pharm Sci. (2002) 5(3):226-30.

1. A method of treating a subject in need of modulating the activity of aldose reductase comprising a step of administering the subject with a pharmaceutical dose of fibrate.

2. The method of claim 1 wherein the fibrate is bezafibrate.

3. The method of claim 1 wherein the fibrate is selected from the group consisting of clofibric acid, ciprofibrate, gemfibrizil, fenofibrate.

4. The method of claim 1 wherein the subject has a condition selected from the group consisting of cardiovascular disease, diabetes, artheriosclerosis, cancer, neoplasm, cataract, retinopathy, keratopathy, nephropathy, neurosis, thrombosis, faulty union of corneal injury and neuropathy.

5. The method of claim 1 further comprising a step of co-administering the subject with a chemotherapeutics.

6. A method of treating a neoplasm comprising a step of contacting a neoplasm cell with fibrate.

7. The method of claim 6 wherein the fibrate is bezafibratethe.

8. The method of claim 6 wherein the fibrate is selected from the group consisting of ciprofibrate, gemfibrizil, fenofibrate.

9. The method of claim 6 wherein the neoplasm is selected from the group consisting of tumor, cancer, fibromas, melanomas, carcinomas, adenocarcinomas, sarcomas, lymphomas, and leukemias.

10. The method of claim 6 further comprising a step of co-contacting the cell with a chemotherapeutics.

11. A method of modulating the activity of aldose reductase in a cell comprising a step of contacting the cell with fibrate.

12. The method of claim 11 wherein the fibrate is bezafibrate.

13. The method of claim 11 wherein the fibrate is selected from the group consisting of clofibric acid, ciprofibrate, gemfibrizil, fenofibrate.
